(19) United States
(12) Patent Application Publication
(10) Pub. No.: US 2018/0265550 A1
(43) Pub. Date: Sep. 20, 2018

(54) THERAPIES, VACCINES, AND PREDICTIVE METHODS FOR FLAVIVIRUSES

(71) Applicants: Samuel Bogoch, New York, NY (US); Elenore S. Bogoch, New York, NY (US); Anne-Elenore Bogoch Borsanyi, New York, NY (US); Samuel Winston Bogoch, Oakland, CA (US)

(72) Inventors: Samuel Bogoch, New York, NY (US); Elenore S. Bogoch, New York, NY (US); Anne-Elenore Bogoch Borsanyi, New York, NY (US); Samuel Winston Bogoch, Oakland, CA (US)

(21) Appl. No.: 15/464,289

(22) Filed: Mar. 20, 2017

(51) Int. Cl. C07K 14/005; C07K 7/06; C07K 7/08; C12N 7/00; C07K 16/10; C12N 15/113; A61K 39/12; G01N 33/68

(52) U.S. Cl. CPC C07K 14/005; C07K 7/06; C07K 7/08; C12N 7/00; C07K 16/10; A61K 2039/505; A61K 39/12; G01N 33/6893; C12N 2770/24122; C12N 2770/24134; C12N 15/1131

(57) ABSTRACT

The present invention provides therapies, vaccines, and predictive methods for Flaviviruses, including Zika virus, Dengue virus, and Japanese encephalitis virus, and provides compounds for diagnosing, preventing, and treating outbreaks of Zika virus, Dengue virus, and Japanese encephalitis virus.

This application incorporates by reference the following applications in their entireties: U.S. Provisional Application Ser. No. 62/287,114, filed Jan. 26, 2016, U.S. application Ser. No. 14/880,210, filed Oct. 10, 2015, U.S. Provisional Application Ser. No. 62/062,859, filed Oct. 11, 2014, U.S. Provisional Appln. Ser. No. 61/993,332, filed May 15, 2014, PCT/US14/34692, filed Apr. 18, 2014, U.S. Provisional Appln. Ser. No. 61/975,674, filed Apr. 4, 2014, PCT/US14/25053, filed Mar. 12, 2014, PCT/US13/69310, filed Feb. 13, 2014, U.S. Provisional Application Ser. No. 61/907,802, filed Nov. 22, 2013, PCT/US13/69310, filed Nov. 8, 2013, U.S. Provisional Application Ser. No. 61/891,677, filed Oct. 16, 2013, PCT/US2013/039111, filed May 1, 2013, U.S. Provisional Application Ser. No. 61/813,889, filed Apr. 19, 2013, U.S. Provisional Application Ser. No. 61/779,324, filed Mar. 13, 2013, U.S. Provisional Application Ser. No. 61/609,074, filed Mar. 9, 2012, U.S. Provisional Appln. Ser. No. 61/765,106, filed Feb. 15, 2013, U.S. Provisional Appln. Ser. No. 61/724,538, filed Nov. 9, 2012, U.S. application Ser. No. 13/553,137, filed Jul. 19, 2012, PCT/US2012/047451, filed Jul. 19, 2012, U.S. Provisional Appln. Ser. No. 61/509,896, filed Jul. 20, 2011, U.S. application Ser. No. 12/581,112, filed Oct. 16, 2009, U.S. Provisional Appln. Ser. No. 61/246,006, filed Sep. 25, 2009, U.S. application Ser. No. 12/538,027, filed Aug. 7, 2009, U.S. Provisional Appln. Ser. No. 61/185,160, filed Jun. 8, 2009, U.S. Provisional Appln. Ser. No. 61/179,686, filed May 19, 2009, U.S. Provisional Appln. Ser. No. 61/172,115, filed Apr. 23, 2009, U.S. application Ser. No. 12/429,044, filed Apr. 23, 2009, and PCT/US09/41565, filed Apr. 23, 2009, U.S. Provisional Appln. Ser. No. 61/143,618, filed Jan. 9, 2009, U.S. Provisional Appln. Ser. No. 61/087,354, filed Aug. 8, 2008, U.S. Provisional Appln. Ser. No. 61/054,010, filed May 16, 2008, U.S. application Ser. No. 12/108,458, filed Apr. 23, 2008, PCT/US2008/61336, filed Apr. 23, 2008, U.S. application Ser. No. 12/010,027, filed Jan. 18, 2008, U.S. Provisional Appln. Ser. No. 60/991,676, filed Nov. 30, 2007, U.S. application Ser. No. 11/923,559, filed Oct. 24, 2007, now U.S. Pat. No. 8,050,871, U.S. Provisional Appln. Ser. No. 60/982,336, filed Oct. 24, 2007, U.S. Provisional Appln. Ser. No. 60/982,333, filed Oct. 24, 2007, U.S. Provisional Appln. Ser. No. 60/982,338, filed Oct. 24, 2007, U.S. Provisional Appln. Ser. No. 60/935,816, filed Aug. 31, 2007, U.S. Provisional Appln. Ser. No. 60/935,499 filed Aug. 16, 2007, U.S. Provisional Appln. Ser. No. 60/954,743, filed Aug. 8, 2007, U.S. application Ser. No. 11/755,597, filed May 30, 2007, U.S. Provisional Appln. Ser. No. 60/898,097, filed Jan. 30, 2007, U.S. Provisional Appln. Ser. No. 60/880,966, filed Jan. 18, 2007, U.S. Provisional Appln. Ser. No. 60/853,744, filed Oct. 24, 2006, U.S. application Ser. No. 11/355,120, filed Feb. 16, 2006, U.S. application Ser. No. 11/116,203, filed Apr. 28, 2005, U.S. application Ser. No. 10/860,050, filed Jun. 4, 2004, now U.S. Pat. No. 7,442,761, U.S. application Ser. No. 10/189,437, filed Jul. 8, 2002, now U.S. Pat. No. 7,452,963, U.S. application Ser. No. 10/105,232, filed Mar. 26, 2002, now U.S. Pat. No. 7,189,800, U.S. application Ser. No. 09/984,057, filed Oct. 26, 2001, now U.S. Pat. No. 7,420,028, and U.S. application Ser. No. 09/984,056, filed Oct. 26, 2001, now U.S. Pat. No. 7,176,275.

### FIELD OF THE INVENTION

The present invention relates to therapies for preventing and treating Flaviviruses, including Zika virus, Dengue virus, and Japanese encephalitis virus, methods of differentiating virulence of strains of Flaviviruses and of predicting outbreaks of Flaviviruses, and compounds for diagnostic, therapeutic, and/or preventive purposes in Flaviviruses.

### BACKGROUND OF THE INVENTION

Flavivirus is a genus of viruses that includes the West Nile virus, dengue virus, tick-borne encephalitis virus, yellow fever virus, Zika virus (ZIKV), Japanese encephalitis virus (JEV), and a handful of other viruses understood to cause encephalitis. Human diseases caused by Flaviviruses include Japanese B encephalitis, Kyasanur Forest disease, St. Louis encephalitis, tick-borne encephalitis, and West Nile encephalitis. Mosquitoes are the most common vector for human disease from Flaviviruses. Some species of virus are tick-borne and some have no known vector.

Flaviviruses are understood to have positive-sense, single stranded RNA genomes of around 10,000-11,000 bases, enveloped, icosahedral nucleocapsid, and range from about 40 to about 65 nm.

Most identified Flaviviruses are transmitted by the bite from an infected mosquito or tick and are classified as arboviruses. Human infections with many Flaviviruses do not produce sufficient titers to be passed back to the vector arthropod. However, at least yellow fever, dengue, and zika viruses (each vectored by a mosquito to humans) apparently do not necessarily depend upon non-human animal hosts.

Zika virus (ZIKV) is a mosquito-born pathogen in humans and others animals. An epidemic of human infection in Brazil in 2015 and 2016 has now been linked to a fetal deformation known as microcephaly in thousands of babies born to mothers infected in Brazil. Infected mothers have born infants with smaller-than-expected brains resulting in brain damage.

Through January 2016, Brazil has reported 3,893 cases of suspected microcephaly. WHO has reported this number to be thirty times more than in any year since 2010. From one to two percent of all newborns in the state of Pernambuco (one of highly infected area) have been identified with microcephaly.

Human infection with Zika virus is generally understood to cause a mild illness in the infected patient. The illness is known as Zika fever or disease and has been compared to mild forms of dengue fever. Current recommended treatment is rest. Until now, it was understood that Zika could not be prevented by drugs or vaccines.

The virus was first identified in a rhesus macaque in Uganda in 1947. It was first isolated from a human in Nigeria in 1968. The virus has been understood to historically be present in Southeast Asia, the Pacific Islands, and parts of Africa.

ZIKV is an icosahedral, enveloped virus with a single-stranded, non-segmented, positive-sense RNA genome. It has been classified in the Flaviviridae virus family and the Flavivirus genus. The virus is understood to be related to the Spondweni virus and has been designated as one of the two viruses in the Spondweni virus clade.

Two lineages of Zika virus have been identified, an African and an Asian lineage. The virus currently identified in Brazil is understood to be closely related to strains previously isolated in French Polynesia.

The positive-sense RNA genome of ZIKV has been identified as having about 10794 bases, including two non-coding flanking regions known as the 5′ NCR and the 3′ NCR. ZIKV open reading frame has been characterized as follows: 5′-C-prM-E-NS1-NS2A-NS2B-NS3-NS4A-NS4B-NS5-3′. The polyprotein coded in the open reading from is understood to be subsequently cleaved into a capsid (C) protein, a precursor membrane (prM) protein, an envelope (E) protein, and non-structural (NS) proteins. The envelope protein comprises a majority of the surface of the virion and is understood to be involved host cell binding and membrane fusion in replication. Non-structural proteins NS 1, NS3, and NS5 have been identified as highly conserved and large. The non-structural proteins NS2A, NS2B, NS4A, and NS4B are understood to be hydrophobic proteins of smaller size. Within in the 3′ NCR 428 nucleotides have been identified. These have been presented as playing a part in translation, RNA packaging, cyclization, genome stabilization, and recognition.

It is understood that the most common Zika virus vector is the female Ae aegypti but also has been isolated from many arboreal mosquito species in the Aedes genus. These include, A. africanus, A. apicoargenteus, A. furcifer, A. hensilli, A. luteocephalus and A. vittatus. Zika virus has also been isolated from Anopheles coustani, Mansonia uniformis, and Culex perfuscus.

The dengue virus (DEN) is the cause of dengue fever and is vectored to humans through mosquitos. At least five serotypes of the virus have been identified as disease-causing in humans and these five serotypes have additionally been considered to exist on a continuum.

The virus genome is around 11000 bases coding for capsid protein C, membrane protein M, envelope protein E (three structural proteins) and NS1, NS2a, NS2b, NS3, NS4a, NS4b, NS5 (seven non-structural proteins) with short non-coding regions on both the 5′ and 3′ ends. Mosquito vectors of DEN include Ae. aegypti and Ae. albopictus,

The Japanese encephalitis virus (JEV) is part of the Japanese encephalitis serocomplex, which is understood to include 9 genetically and antigenically related viruses. Members of this complex are particularly severe in horses, and four are known to infect humans, including West Nile virus.

Wild birds (including herons) and pigs are understood to be reservoirs of the virus. Infection in humans may be severe. Vectors of JEV include the mosquitoes Culex tritaeniorhynchus and Culex vishnui.

There is a need in the art for quantitative methods of predicting further progression and additional outbreaks or waning of Flaviviruses, including ZIKV, DEN, and JEV. There is likewise a need in the art for methods of preventing, and treating ZIKV, DEN, and JEV infections and outbreaks and a need for therapies against ZIKV, DEN, and JEV including prophylactic therapies, such as vaccines, and treatments, such as therapies for providing passive immunity and other methods of blocking progression or transmission of ZIKV, DEN, and JEV.

Replikin peptides are a family of small peptides that have been correlated with the phenomenon of rapid replication in SARS, influenza, malaria, West Nile virus, foot and mouth disease, and many other pathogens. See, e.g., WO 2008/143717. Replikin peptides have likewise been generally correlated with the phenomenon of rapid replication in viruses, organisms, and malignancies.

Identification of Replikin peptides has provided targets for detection and treatment of pathogens, including vaccine development against virulent pathogens such as influenza virus, malaria, West Nile virus, and foot and mouth disease virus. See, e.g., WO 2008/143717. In general, knowledge of and identification of this family of peptides enables development of effective therapies and vaccines for pathogens that harbor Replikins. The phenomenon of the association of Replikins with rapid replication and virulence has been fully described in U.S. Pat. No. 7,189,800; U.S. Pat. No. 7,176,275; U.S. Pat. No. 7,442,761; U.S. Pat. No. 7,894,999, U.S. Pat. No. 8,050,871, and US 2009/0041795. Both Replikin concentration (number of Replikins per 100 amino acids) and Replikin composition have been correlated with the functional phenomenon of rapid replication.

There is a continuing need for monitoring Replikin sequences in ZIKV to identify compounds for therapies that target ZIKY. There is also a need to develop Replikin-based therapies that are effective across strains and within strains as they mutate over time.

In response to these continuing needs and despite extensive efforts in the art to understand infectivity and virulence in ZIKV, and to track and predict outbreaks of ZIKV, applicants have now surprisingly applied their previous discovery of Replikin chemistry in the virus genome structure to methods of predicting outbreaks of ZIKV in real time. They have likewise now surprisingly provided methods of identifying conserved targets in emerging strains of ZIKV against which pharmaceutical compositions and vaccines are provided and likewise may be provided prior to or at the outset of any further outbreak. Genome analysis may be undertaken in real time and vaccine may be produced in as few as seven days.

### SUMMARY OF THE INVENTION

The present invention provides compounds for diagnostic, therapeutic, and/or preventive purposes against Flaviviruses and methods of predicting outbreaks of Flaviviruses.

The present invention likewise provides compounds for diagnostic, therapeutic, and/or preventive purposes against Zika virus (ZIKV), Dengue virus (DEN), and Japanese Encephalitis virus (JEV) and methods of predicting outbreaks of ZIKV, DEN, and JEV (among other Flaviviruses).

A first non-limiting aspect of the present invention provides an isolated or synthesized peptide consisting of no more than 50 amino acid residues comprising a Replikin sequence from an isolate of ZIKV or a homologue sharing at least 50%, 60%, 70%, 80%, 90%, or 95% or more homology with a Replikin sequence from an isolate of ZIKV, an isolate of DEN or a homologue sharing at least 50%, 60%, 70%, 80%, 90%, or 95% or more homology with a Replikin sequence from an isolate of DEN, or an isolate of JEV or a homologue sharing at least 50%, 60%, 70%, 80%, 90%, or 95% or more homology with a Replikin sequence from an isolate of ZIKY.

In a non-limiting embodiment, the Replikin sequence is identified as shared among isolates of at least two of ZIKV, DEN, and JEV or homologues of the Replikin sequence are identified as shared among isolates of at least two of ZIKV, DEN, and JEV, wherein said homologues share at least 50%, 60%, 70%, 80%, 90%, or 95% or more homology.

In a non-limiting embodiment, the Replikin sequence is identified as shared among isolates of all three of ZIKV, DEN, and JEV or homologues of the Replikin sequence are shared among all three of ZIKV, DEN, and JEV, wherein said homologues share at least 50%, 60%, 70%, 80%, 90%, or 95% or more homology.

A non-limiting embodiment of the first aspect of the invention provides a composition comprising at least one peptide consisting of no more than 50 amino acid residues comprising a Replikin sequence from an isolate of ZIKV, DEN, or JEV or a homologue sharing at least 50%, 60%, 70%, 80%, 90%, or 95% or more homology with a Replikin sequence from an isolate of ZIKV, DEN, or JEV. In a non-limiting embodiment, the composition is a pharmaceutical composition. In a non-limiting embodiment, the pharmaceutical composition is a blocking composition. In a non-limiting embodiment, the pharmaceutical composition is an immunogenic composition. In a non-limiting embodiment, the pharmaceutical composition further comprises a pharmaceutically-acceptable carrier, excipient, and/or adjuvant.

In a non-limiting embodiment, the Replikin sequence is identified as shared among isolates of at least two of ZIKV, DEN, and JEV or homologues of the Replikin sequence are identified as shared among isolates of at least two of ZIKV, DEN, and JEV. In a non-limiting embodiment, the Replikin sequence is identified as shared among isolates of all three of ZIKV, DEN, and JEV or homologues of the Replikin sequence are shared among all three of ZIKV, DEN, and JEV. In a non-limiting embodiment, the Replikin sequence is SEQ ID NO: 5.

In a non-limiting embodiment of the first aspect of the present invention, a composition may comprise at least two peptides, each consisting of no more than 50 amino acid residues comprising a Replikin sequence from an isolate of ZIKV, DEN, or JEV or a homologue sharing at least 50%, 60%, 70%, 80%, 90%, or 95% or more homology with a Replikin sequence from an isolate of ZIKV, DEN, or JEV, wherein that at least two peptides are covalently linked. In a non-limiting embodiment, the composition may comprise at least three or more peptides that are covalently linked. In a non-limiting embodiment, covalent linkage may be through a peptide bond. In another non-limiting embodiment, the linkage may be through PEGylation. In a further non-limiting embodiment, linkage may be through an Ahx spacer. Linkage may be done by any method known to one of skill in the art now or hereafter.

A non-limiting embodiment of the first aspect of the invention provides at least one peptide consisting of no more than 50 amino acid residues comprising at least one sequence of anyone of SEQ ID NO(s): 1-7 or at least one homologue sharing at least 50%, 60%, 70%, 80%, 90%, or 95% or more homology with anyone of SEQ ID NO(s): 1-7. In a non-limiting embodiment, the at least one peptide consists essentially of anyone of SEQ ID NO(s): 1-7. In a non-limiting embodiment, the at least one peptide consists of anyone of SEQ ID NO(s): 1-7. In a non-limiting embodiment, the at least one peptide consists of a homologue of anyone of SEQ ID NO(s): 1-7, wherein said homologue shares at least 50%, 60%, 70%, 80%, 90%, or 95% or more homology with anyone of SEQ ID NO(s): 1-7. In a non-limiting embodiment, a composition may comprise at least one peptide consisting of no more than 50 amino acid residues comprising at least one sequence of anyone of SEQ ID NO(s): 1-7 or at least one homologue sharing at least 50%, 60%, 70%, 80%, 90%, or 95% or more homology with anyone of SEQ ID NO(s): 1-7.

A non-limiting embodiment of the first aspect of the invention provides at least one described peptide that is lyophilized. A non-limiting embodiment of the first aspect of the invention provides at least one described peptide that is freeze-dried. A non-limiting embodiment of the first aspect of the invention provides at least one described peptide that is dissolved in an aqueous solution. The aqueous solution may be pure deionized water or may be sterilized water.

A non-limiting embodiment of the first aspect of the invention provides a pharmaceutical composition administrable to a subject susceptible to or suffering from a ZIKV infection, said composition comprising at least one peptide consisting of no more than 50 amino acid residues comprising a sequence of anyone of SEQ ID NO(s): 1-7 or homologue thereof and a pharmaceutically-acceptable carrier or adjuvant.

A non-limiting embodiment of the first aspect of the invention provides a pharmaceutical composition comprising a mixture of at least two peptides each consisting of no more than 50 amino acid residues, wherein each of said two peptide comprises at least one amino acid sequence of SEQ ID NO(s): 1-7 that is different from the other of said two peptides. In a non-limiting embodiment, a pharmaceutical composition comprises a mixture of at least three, four, five, six, or seven or more different peptides of SEQ ID NO(s): 1-7.

In a non-limiting embodiment of the first aspect of the invention, administration of a pharmaceutical composition to a subject having an immune system stimulates an immune response against anyone of the amino acid sequences of SEQ ID NO(s): 1-7.

A non-limiting second aspect of the present invention provides a vaccine against a Flavivirus. In a non-limiting embodiment, the vaccine is against at least ZIKV, DEN, or JEV. In a non-limiting embodiment, the vaccine is against at least two of ZIKV, DEN, or JEV. In a non-limiting embodiment, the vaccine is against all three of ZIKV, DEN, or JEV. In a non-limiting embodiment, the vaccine comprises a pharmaceutical composition of the first aspect of the invention.

A non-limiting third aspect of the present invention provides a method of preventing or treating a ZIKV, DEN, or JEV infection comprising administering a pharmaceutical composition of the first non-limiting aspect of the invention or a vaccine of the second non-limiting aspect of the invention to a subject susceptible to or suffering from a ZIKV, DEN, or JEV infection. In a non-limiting embodiment, administration of the pharmaceutical composition or vaccine stimulates an immune response in the subject against a ZIKV, DEN, or JEV. In a non-limiting embodiment, administration of the pharmaceutical composition or vaccine provides a blocking effect against a ZIKV, DEN, or JEV.

A non-limiting fourth aspect of the present invention provides a method of stimulating the immune system of a subject against ZIKV, DEN, or JEV comprising administering a pharmaceutical composition of the first aspect of the invention or a vaccine of the second aspect of the invention to the subject. In a non-limiting embodiment, the subject is suitable for providing antibodies against a ZIKV, DEN, or JEV. In a non-limiting embodiment, the subject is suitable for production of polyclonal antibodies. In a non-limiting embodiment, the subject is suitable for production of monoclonal antibodies. In a non-limiting embodiment, the polyclonal antibodies or monoclonal antibodies are useful for providing passive immunity in a different subject. In a non-limiting embodiment, the polyclonal antibodies or monoclonal antibodies are useful for identifying ZIKV, DEN, or JEV and/or are useful for diagnosing ZIKV, DEN or JEV.

A non-limiting fifth aspect of the present invention provides an isolated, chemically-synthesized, or recombinantly-generated binding molecule that specifically binds a Replikin sequence of a ZIKV, DEN, or JEV and/or that specifically binds a homologue of a Replikin sequence of a ZIKV, DEN, or JEV, wherein the homologue is at least 50%, 60%, 70%, 80%, 90%, 95%, or more homologous with a Replikin sequence of a ZIKV, DEN, or JEV.

In a non-limiting embodiment of the fifth aspect of the present invention, the binding molecule binds at least one sequence of SEQ ID NO(s): 1-7 or a homologue of at least one sequence of SEQ ID NO(s): 1-7, wherein said homologue is at least 50%, 60%, 70%, 80%, 90%, 95%, or more homologous.

In a non-limiting embodiment, the binding molecule is an antibody or antibody fragment. In a non-limiting embodiment, the antibody is a monoclonal antibody. In a nonlimiting embodiment, the antibody is a humanized antibody. In a non-limiting embodiment, the antibody is an optimized antibody. In a non-limiting embodiment, the antibody is an fc-optimized antibody. In a non-limiting embodiment, the antibody is selected using phage display. In a non-limiting embodiment, the antibody is produced from a microorganism such as E. coli.

A non-limiting sixth aspect of the present invention provides methods of delivering passive immunity to a patient suffering from a ZIKV, DEN, or JEV infection comprising administering to the patient at least one isolated, chemically-synthesized, or recombinantly-generated binding molecule of the fifth aspect of the invention.

A non-limiting seventh aspect of the present invention provides a method of making a pharmaceutical composition comprising: selecting at least one isolated or synthesized protein, protein fragment, polypeptide, or peptide comprising at least one peptide sequence that is at least 30%, 40%, 50%, 60%, 70%, 80%, 90% or 95%, or 100% homologous with at least one Replikin peptide sequence identified in a ZIKV, DEN, or JEV as a component of a pharmaceutical composition; and making said pharmaceutical composition. In a non-limiting embodiment, the at least one Replikin peptide sequence is identified as shared among at least two of ZIKV, DEN, and JEV. In a non-limiting embodiment, the at least one Replikin peptide sequence is identified as shared among all three of ZIKV, DEN, and JEV. In a non-limiting embodiment, a Replikin sequence shared among ZIKV, DEN, and/or JEV is a homologue that is at least 50%, 60%, 70%, 80%, 90%, or 95% homologous.

In a non-limiting embodiment, the method of making a pharmaceutical composition comprises selecting at least one isolated or synthesized peptide of SEQ ID NO(s): 1-7, as at least one component, and making said vaccine with the at least one component. In a non-limiting embodiment, the isolated or synthesized peptide comprises anyone of SEQ ID NO(s): 1-7 and is up to 50, 60, 70, 80, 90, 100, 150, 200, or 250 residues in length. In a non-limiting embodiment, the pharmaceutical composition is comprised in a vaccine.

In another non-limiting embodiment, a method of making a pharmaceutical composition comprises selecting at least two, three, four, five, six, seven, eight, nine, ten, eleven, twelve, thirteen, fourteen, fifteen, sixteen, seventeen, or twenty or more isolated or synthesized Replikin peptide sequences identified in ZIKV, DEN, or JEV and/or isolated or synthesized functional fragments of Replikin peptide sequences identified in ZIKV, DEN, or JEV and/or isolated or synthesized homologues of Replikin peptide sequences identified in ZIKV, DEN, or JEV and making a pharmaceutical composition comprising said selected peptide sequences or functional fragments thereof. In a further embodiment, the isolated or synthesized Replikin peptide sequences, homologs, or functional fragments thereof comprise at least one peptide sequence of SEQ ID NO(s): 1-7, at least one functional fragment of at least one peptide sequence of SEQ ID NO(s): 1-7, or at least one functional fragment of at least one Replikin peptide sequence identified in a ZIKV, DEN, or JEV and may consist of said sequences and may comprise said sequences and be up to 50, 60, 70, 80, 90, 100, 150, 200, 250 or more residues in length. In another non-limiting embodiment, the at least one isolated or synthesized protein, protein fragment, polypeptide, or peptide has the same amino acid sequence as at least one protein, protein fragment, polypeptide or peptide identified in a relatively virulent strain of ZIKV up to seven days, one month, six months, one year, two years, or three years prior to making said vaccine.

An eighth non-limiting aspect of the present invention provides a method of determining an increased probability of an outbreak of ZIKV, DEN, or JEV within about six months to about one year following an increase in Replikin concentration in an isolate of ZIKV, DEN, or JEV comprising identifying an increase in the concentration of Replikin sequences in at least one first isolate of ZIKV, DEN, or JEV as compared to at least one other isolate of ZIKV, DEN, or JEV wherein said at least one first isolate is isolated at a later time period than said one other isolate and wherein said increase in the concentration of Replikin sequences signifies the increased probability of the outbreak of ZIKV, DEN, or JEV within about six months to about one year following the increase in the concentration of Replikin sequences.

In a non-limiting embodiment, a method of prediction comprises: (1) obtaining a plurality of isolates of a ZIKV, DEN, or JEV wherein at least one of said isolates is isolated about six months to about 3 years later than at least one other of said isolates; (2) analyzing the amino acid sequence of at least one protein or protein fragment in each isolate of the plurality of isolates for the presence and concentration of Replikin sequences; (3) comparing the concentrations of Replikin sequences in the at least one protein or protein fragment in each isolate of the plurality of isolates one to another; (4) identifying an increase in the concentration of Replikin sequences in said plurality of isolates over at least one time period of about six months or greater; and (5) predicting an outbreak of the ZIKV, DEN, or JEV within about one month to about three years following said identified increase in the concentration of Replikin sequences. In another embodiment of the invention, the ZIKV, DEN, or JEV outbreak is predicted within about six months. In a further embodiment, the ZIKV, DEN, or JEV outbreak is predicted within about one year to about three years. In a further non-limiting embodiment, the method of prediction further comprises processing at least one step of the method on a computer.

An ninth non-limiting aspect of the present invention provides a nucleic acid sequence that is antisense to a nucleic acid that encodes for any Replikin peptide present in or identified in an isolate ZIKV, DEN, or JEV. This may include one of SEQ ID NO(s): 1-7 or a small interfering nucleic acid sequence that interferes with a nucleic acid sequence that is 30%, 40%, 50%, 60%, 70%, 80%, 90%, or 95% or more homologous with a nucleic acid that encodes any Replikin peptide of a ZIKV, DEN, or JEV including, for example, any one of SEQ ID NO(s): 1-7 or is 30%, 40%, 50%, 60%, 70%, 80%, 90%, 95% or more homologous with a nucleic acid that is antisense to a nucleic acid that encodes for anyone of SEQ ID NO(s): 1-7. In a non-limiting embodiment, the nucleic acid sequence is 21 to 150 nucleotides in length. In a non-limiting embodiment, the nucleic acid sequence is up to 5, 10, 15, 20, 25, 30, 35, 40, 45, or 50 nucleotides in length. In a non-limiting embodiment, the nucleic acid encodes for or is antisense to a nucleic acid that encodes for a homologue of a Replikin peptide of ZIKV, DEN, or JEV.

### BRIEF DESCRIPTION OF THE DRAWINGS

FIG. 1 illustrates the annual mean Replikin concentration of all Zika virus sequences from animal and human subjects available to applicants in a given year when queried at the NCBI PubMed database website for isolates from 1947 through 2015 and number of microcephaly cases reported from 2010 through January 2016. For determination of mean Replikin concentration, 179 sequences were available in the NCBI database when queried. Dark blue lines reflect mean Replikin concentration. Light blue lines reflect standard deviation from the annual mean. The two dark blue lines with light blue standard deviation illustrated in 2015 are from queries through May 2015 and through October 2015. Dark red lines from 2010 through 2016 illustrate cases of reported microcephaly times 200. An increase in Replikin concentration in isolates of ZIKV reported to the PubMed NCBI database in May and October of 2015 correlates with the outbreak of ZIKV and incidence of microcephaly in Brazil.

FIG. 2 illustrates the number of appearance over time of SEQ ID NO: 5, a particular, single, 100% identical, Replikin sequence encoded in the genes of three related Flaviviruses, namely, Zika virus (ZIKV), Dengue virus (DEN), and Japanese Encephalitis virus (JEV) as reported in the NCBI PubMed database in the given year. In the figure, the blue lines represent the number of isolates of JEV identified as having the SEQ ID NO: 5 Replikin sequence encoded in the genome, the green lines represent the number of isolates of DEN identified as having the SEQ ID NO: 5 Replikin sequence encoded in the genome, and the black lines represent the number of isolates (times 10) of ZIKV identified as having the SEQ ID NO: 5 Replikin sequence encoded in the genome.

### DETAILED DESCRIPTION OF THE INVENTION

### Definitions

A “protein fragment” as used in this specification is any fragment of an expressed whole protein, which is any portion of an expressed whole protein where a “portion” of a protein is less than an expressed whole protein. A protein fragment is not naturally produced as part of the biological function of the virus. A protein fragment reflects an expressed whole protein with one or more amino acids removed from the amino acid sequence of the expressed whole protein. A protein fragment may also reflect an amino acid sequence that is at least 30%, 40%, 50%, 60%, 70%, 80%, 90%, 95%, or more homologous with any portion of an expressed whole protein so long as the protein fragment does not reflect the entire amino acid sequence of the expressed whole protein or the functional protein following biological processing. A “polypeptide,” as used in this specification, is any portion of a protein fragment and is less than an expressed whole protein. A protein fragment may reflect 50, 100, 150, 200, or more amino acid residues (or any size in between) so long as the protein fragment reflects less than the total length of the expressed whole protein.

A “functional fragment” of a Replikin sequence as described herein is a fragment, variant, analog, or chemical derivative of a Replikin sequence that retains at least a portion of the immunological cross reactivity with an antibody specific for the Replikin sequence. A fragment of the Replikin sequence refers to any subset of the molecule. Variant peptides of the sequence may be made by direct chemical synthesis, for example, using methods well known in the art. An analog of a Replikin sequence to a non-natural protein or polypeptide is substantially similar to either the Replikin sequence of the protein or a fragment thereof. Chemical derivatives of a Replikin sequence contain additional chemical moieties. Amino acid analogues may include D-amino acid residues, or polypeptides with modified structural backbones, or polypeptides with non-natural chemical moieties added.

As used herein, the term “preferentially binds” or “specifically binds” and related terms referencing the interaction of a binding molecule such as, for example, an antibody, and the structure to which it binds (antigen) means that the binding molecule preferentially recognizes the structure to which it binds even when present among other molecules (such as in a mixture of molecules). Specific or preferential binding of a binding molecule to a binding structure or an immunogenic portion of a binding structure is specific and preferential when the binding molecule binds to the structure or portion thereof and does not bind with the same level of affinity to other structures. Binding affinity may be determined by one of ordinary skill in the art using, for example, BIACORE, enzyme-linked immunosorbent assays, or radioimmuno assays. A binding molecule may cross-react with related antigens and preferably does not crossreact with affinity to unrelated antigens. Binding between a binding molecule and the structure to which it binds may be mediated by covalent or non-covalent attachment, or both.

As used herein a “vaccine” is any substance, compound, composition, mixture, or other therapeutic substance that, when administered to a human or animal via any method of administration known to the skilled artisan now or hereafter, produces an immune response, an antibody response, or a protective effect in the human or animal.

As used herein, a “Replikin sequence” is an amino acid sequence of 7 to 50 amino acid residues comprising (1) a first lysine residue located six to ten residues from a second lysine residue; (2) at least one histidine residue; and (3) at least 6% lysine residues, where the sequence is the shortest sequence comprising the first and second lysine residues of element (1) and the at least one histidine of element (2). A Replikin sequence may comprise more than two lysine residues and more than one histidine residue so long as at least two of the lysine residues and at least one histidine residue reflects the requirements of the definition of a Replikin sequence. For diagnostic, therapeutic, and preventive purposes, a Replikin sequence mayor may not be the shortest sequence comprising the first and second lysine residues of element (1) and the at least one histidine residue of element (2). A Replikin sequence may comprise a terminal lysine residue and a terminal lysine or histidine residue where the sequence is 7 to 50 amino acid residues in length and comprises (1) at least one lysine residue located six to ten residues from at least one other lysine residue; (2) at least one histidine residue; and (3) at least 6% lysine residues.

The term “Replikin sequence” can also refer to a nucleic acid sequence encoding an amino acid sequence having 7 to about 50 amino acids comprising:
(1) at least one lysine residue located six to ten amino acid residues from a second lysine residue;
(2) at least one histidine residue; and
(3) at least 6% lysine residues.

As used herein, an “isolated” peptide may be synthesized by organic chemical methods. An isolated peptide may also be synthesized by biosynthetic methods. An isolated peptide may also refer to a peptide that is, after purification, substantially free of cellular material or other contaminating proteins or peptides from the cell or tissue source from which the peptide is derived, or substantially free from chemical precursors or other chemicals when chemically synthesized by any method, or substantially free from contaminating peptides when synthesized by recombinant gene techniques or a protein or peptide that has been isolated in silica from nucleic acid or amino acid sequences that are available through public or private databases or sequence collections. An isolated peptide may be synthesized by biosynthetic or organic chemical methods.

Proteins, protein fragments, polypeptides, or peptides in this specification may be chemically synthesized by any method known to one of skill in the art now and hereafter. For example, isolated proteins, protein fragments, polypeptides, or peptides may be synthesized by solid phase synthesis. The production of these materials by chemical synthesis avoids the inclusion of (or the need to remove by purification) materials that are byproducts of other production methods such as recombinant expression or isolation from biological material. Such byproducts may include, for example, avian proteins associated with vaccines produced using birds' eggs, bacterial proteins associated with recombinant production in bacteria, or proteins or contaminants associated with any recombinant activity such as with productions of proteins or other sequences in insect cells.

An “encoded” or “expressed” protein, protein sequence, protein fragment sequence, or peptide sequence is a sequence encoded by a nucleic acid sequence that encodes the amino acids of the protein or peptide sequence with any codon known to one of ordinary skill in the art now or hereafter. It should be noted that it is well known in the art that, due to redundancy in the genetic code, individual nucleotides can be readily exchanged in a codon and still result in an identical amino acid sequence. As will be understood by one of ordinary skill in the art, a method of identifying a Replikin amino acid sequence also encompasses a method of identifying a nucleic acid sequence that encodes a Replikin amino acid sequence wherein the Replikin amino acid sequence is encoded by the identified nucleic acid sequence.

“Homologous” or “homology” or “sequence identity” as used in this specification indicate that an amino acid sequence or nucleic acid sequence exhibits substantial structural equivalence with another sequence, namely any Replikin peptide sequence (including SEQ ID NO(s): 1-7) identified in an isolate of ZIKV or any nucleotide sequence encoding a Replikin peptide sequence in an isolate of ZIKV (a redundancy in a coding sequence may be considered identical to a sequence encoding the same amino acid). To determine the percent identity or percent homology of an identified sequence, a sequence is aligned for optimal comparison purposes with anyone of possible basis sequences. For purposes of this paragraph, a basis sequence is a Replikin sequence identified in an isolate of ZIKY. Where gaps are necessary to provide optimal alignment, gaps may be introduced in the identified sequence or in the basis sequence. When a position in the identified sequence is occupied by the same amino acid residue or same nucleotide as the corresponding position in the basis sequence, the molecules are considered identical at that position (as used herein amino acid or nucleic acid “identity” is equivalent to amino acid or nucleic acid “homology”). To determine percent homology, the amino acid residues or nucleotides at corresponding amino acid positions or nucleotide positions are compared between the identified sequence and the basis sequence. The total number of amino acid residues or nucleotides in the identified sequence that are identical with amino acid residues or nucleotides in the basis sequence is divided by the total number of residues or nucleotides in the basis sequence (if the number of residues or nucleotides in the basis sequence is greater than the total number of residues or nucleotides in the identified sequence) or by the total number of amino acid residues or nucleotides in the identified sequence (if the number of residues or nucleotides in the identified sequence is greater than the total number of residues or nucleotides in the basis sequence). The final number is determined as a percentage. As such, the percent identity between the two sequences is a function of the number of identical positions shared by the sequences, taking into account the number of gaps (where a gap must be introduced for optimal alignment of the two sequences) and the length of each gap. Any structural or functional differences between sequences having sequence identity or homology will not affect the ability of the sequence to function as indicated in the desired application.

For example, SEQ ID NO: 8 (KHATVLK) is considered 70% homologous with SEQ ID NO: 9 (KVKKHATVLK). The 70% homology between SEQ ID NO: 8 and SEQ ID NO: 9 is determined as follows: SEQ ID NO: 8 is the identified sequence. SEQ ID NO: 9 is the basis sequence. Upon alignment, SEQ ID NO: 8 is identical to SEQ ID NO: 9 in seven of the ten sequences of SEQ ID NO: 9. To determine percent homology, then, the 7 aligned identical residues are divided by the total number of residues in SEQ ID NO: 9, namely 10 residues, giving 0.70 or more than 70% homology.

To determine homology between an identified sequence that is contained in a larger polypeptide, protein fragment, or protein, and a basis sequence, the polypeptide, protein fragment, or protein must first be optimally aligned with the basis sequence. Upon alignment of the sequences, the residue in the identified sequence that is furthest to the amino-terminus of the polypeptide, protein fragment, or protein and identical to a residue in the basis sequence that is furthest to the amino-terminus of the basis sequence is considered the amino-terminal residue of the identified sequence. Likewise, upon alignment, the residue in the identified sequence that is furthest to the carboxy-terminus of the polypeptide, protein fragment, or protein and identical to a residue in the basis sequence that is furthest to the carboxy-terminus of the basis sequence is considered the carboxy-terminal residue of the identified sequence.

Concerning gaps, the number of gaps in either the basis sequence or the identified sequence should be limited to the number of gaps allowable without significantly compromising the function of the identified sequence as compared to the basis sequence. In general, many gaps in the sequence of the basis peptide or in the sequence of the identified peptide are allowed based on homology as defined herein. Relatively more gaps are allowed if the lysines and histidines that create the definition of the Replikin peptide are identically shared between the basis peptide and the identified peptide. Relatively more gaps are also allowed if the lysines and histidines that create the definition of the Replikin peptide are shared at least in close position (for example within ten, nine, eight, seven, six, five, four, three, two, or one amino acid residue). If some of the lysine residues and histidine residues that create the definition of the Replikin peptide are not present in the identified peptide, fewer gaps may be allowed. Nevertheless, if the identified peptide functions similarly to the basis peptide, any number of gaps is allowed. In general, three or more gaps are allowed in the sequence of the basis peptide or in the sequence of the identified peptide within ten amino acid residues of the basis peptide if no lysines or histidines are present in the identified peptide. Two or more gaps or one or more gaps are also allowed. Nevertheless, if the identified sequence provides the same or a similar function to the basis sequence, more gaps are allowed up to the number of gaps that will provide a homology of 30%, 40%, 50%, 60%, 70%, 80%, 90%, 95%, or more homology. Additionally, where the lysines and histidines of the Replikin definition are present in both the identified peptide and the basis peptide, there should be no limit on how many gaps are allowed.

The presence of lysines and histidines providing for the Replikin definition in an identified peptide requires significantly less homology because the lysines and the histidines of the Replikin definition provide for conservation of Replikin function. For example, in Table 8 and the description thereof in columns 62 and 63 in U.S. Pat. No. 7,442,761, a highly mutable tat protein in HIV is described and analyzed. As may be seen from Table 8 in U.S. Pat. No. 7,442,761, in tat protein of HIV, which is essential for replication in the virus, lysines and histidines that are essential to maintaining the Replikin definition within a key Replikin peptide in the protein are observed to be 100% conserved, while substitutions in amino acid residues that are not essential to maintaining the Replikin definition are commonly substituted. The conservation of the key amino acids for maintaining the Replikin definition is understood to provide a specific survival function for HIY. The same phenomenon is seen in influenza. See U.S. Pat. No. 7,442,761, column 62, lines 42-45. This phenomenon has now also been surprisingly identified in ZIKY. See, e.g., Table 12.

As used herein, “Replikin Count” or “Replikin concentration” refers to the number of Replikin sequences per 100 amino acids in a protein, protein fragment, virus, or organism. A higher Replikin concentration in a first strain of a virus or organism has been found to correlate with more rapid replication of the first virus or organism as compared to a second, earlier-arising or later-arising strain of the virus or organism having a lower Replikin concentration. Replikin concentration is determined by counting the number of Replikin sequences in a given sequence, wherein a Replikin sequence is a peptide of 7 to 50 amino acid residues comprising (1) a first lysine residue six to ten residues from a second lysine residue, (2) at least one histidine residue, (3) and 6% or more lysine residues where the Replikin sequence is the shortest sequence comprising the first and second lysine residues of element (1) and the at least one histidine residue of element (2). A Replikin sequence may comprise more than two lysine residues and more than one histidine residue so long as there is at least one lysine residue six to ten residues from a second lysine residue and at least one histidine residue. A Replikin sequence for the purpose of determining Replikin concentration as described in this paragraph may also be a nucleic acid that encodes a Replikin peptide sequence defined according to this paragraph.

### Pharmaceutical Compositions

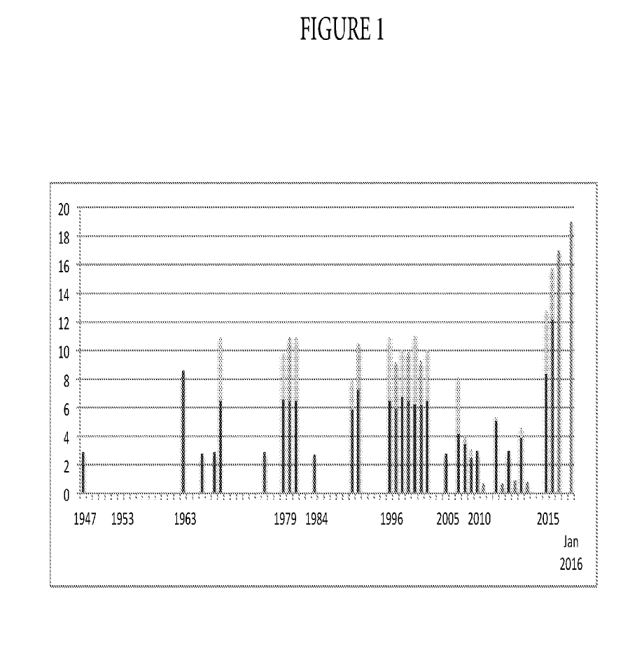

As illustrated in FIG. 1, Zika viruses are structurally and functionally related to rapid replication, virulence, and viral outbreaks. Further, Examples 2 through 8 demonstrate that Replikin sequences are highly conserved across time and regions. Identification of these structures, therefore, allows for targeting of the function of rapid replication (related to virulence) in ZIKV by stimulating an immune response against these functional targets and by administering blocking compounds (including Replikin sequences, binding molecules against Replikin sequences, and antisense nucleic acid sequences against Replikin sequences) that interfere with these functional targets.

One non-limiting aspect of the invention, therefore, provides compounds and compositions that stimulate an immune response against or otherwise mechanically block Replikin sequences resulting in inhibition of replication in the virus and diminishment of virulence. Pharmaceutical compositions that target highly-conserved sequences and/or sequences present in a current outbreak are useful for treating and preventing ZIKY.

Compounds and the active part of said compositions may be produced, for example, by solid-phase synthesis of peptide sequences. These peptide sequences may be freeze dried, or lyophilized, providing for long term stability of the peptides without refrigeration or other stabilizers or preservatives. Because the peptides are short, they are soluble in water and may be dissolved in water for administration as a vaccine or blocking agent without additional solubilizing compounds. These compositions need not contain biologics and need not be refrigerated. Production may be ramped up easily with economies of scale to meet large populations (even the global population). Compositions may be manufactured in as little as seven days. Studies in influenza and taura syndrome virus have demonstrated both an immune response and blocking responses. See, e.g., US 2009/0041795 and US 2010/0215675 (each incorporated herein by reference).

Administration of a pharmaceutical composition to a subject having an immune system is understood to result in an immune response and/or blocking response against Replikin sequences in ZIKV including the amino acid sequences of SEQ ID NO(s): 1-7 or homologues thereof. As a result, the pharmaceutical composition is useful for targeting replication and virulence in ZIKY.

Use of a herein-described peptide sequence for the manufacture of a pharmaceutical composition against ZIKV is provided. Use of a herein-described pharmaceutical composition for administration to an animal to provide a blocking response and/or an immune response and a resulting protective effect against ZIKV infection is provided.

### Blocking and Immunogenic Compounds

A non-limiting immunogenic and/or blocking compound is provided comprising at least one protein, protein fragment, polypeptide, or peptide of anyone of the proteins, protein fragments, polypeptides, or peptides described herein including and not limited to comprising at least one Replikin peptide sequence identified in a ZIKV or at least one homologue of said at least one Replikin peptide sequence, or at least one functional fragment of at least one Replikin peptide sequence.

A Replikin sequence may be shared among different isolates of ZIKY. The sequence may be shared among any two or more of isolates of ZIKY. The Replikin sequences may be shared as identical or as close homologues. The Replikin sequences may differ by a single amino acid residue. The Replikin sequences may be conserved in ZIKV over one, two, three, or more years.

Replikin sequences identified in ZIKV may be altered by a single amino acid sequence to create a sequence not present in ZIKV in nature. Replikin peptide sequences may be covalently linked to other Replikin sequences, such as anyone or more of SEQ ID NO(s): 1-7. Peptidomimetic versions of Replikin peptide sequences or homologues thereof may likewise be used for immunogenic or blocking compositions. Poly-N-substituted glycines, D-peptides, or beta-peptides may be constructed to function as any identified Replikin peptide sequence. Linkage may be by any method known to art now or hereafter including but not limited to PEGylation, peptoid bonds, Ahx spacers, etc.

### Preventing and Treating ZIKV Infections

Because Replikin sequences may be targeted to inhibit or control replication and virulence in ZIKY. A method of preventing or treating ZIKV infection is provided comprising administering a pharmaceutical composition or a vaccine comprising a immunological and/or blocking composition to a subject susceptible to or suffering from a ZIKV infection. Administration of a pharmaceutical composition or vaccine stimulates an immune response in the subject against ZIKY. Administration of a pharmaceutical composition or vaccine provides a blocking response against ZIKY.

The pharmaceutical compositions of the invention, alone or in various combinations, may be administered to a subject by any manner known to one of ordinary skill in the art including by intravenous or intramuscular injection, ocular swab or spray, nasal spray and/or inhalation spray, or any other method of administration in order to stimulate the immune system of the subject to produce an immune response or in order to provide a direct or otherwise indirect blocking effect. Generally the dosage of peptides in a pharmaceutical composition is in the range of from about 0.1 μg to about 10 mg, about 10 μg to about 1 mg, and about 50 μg to about 500 μg. A non-limiting dosage may be 0.01 to 5 mg of peptide per gram of body mass of a subject. A non-limiting dosage may be 0.05 to 1 mg of peptide per gram of body mass of a subject. A non-limiting dosage may be 0.1 to 0.5 mg of peptide per gram of body mass of a subject. The skilled practitioner can readily determine the dosage and number of doses needed to produce an effective immune response or an effective blocking effect, or both.

### Stimulating Immune Responses

Replikin sequences and peptides have been shown to be highly immunogenic where antibodies produced against the sequences or peptides target replication. See, e.g., US 2003/0194414 (incorporated herein by reference). The identification herein of function Replikin sequences conserved in ZIKV provides a method of stimulating the immune system of a subject against ZIKV comprising administering a pharmaceutical composition or a vaccine to a subject. The subject may be a suitable subject for providing antibodies against ZIKY. The subject may be suitable for stimulation and production of polyclonal antibodies or for stimulation and production of monoclonal antibodies. The polyclonal antibodies and monoclonal antibodies are useful for providing passive immunity in a patient. The polyclonal antibodies and monoclonal antibodies are likewise useful for identifying ZIKV and/or are useful for diagnosing ZIKY.

### Vaccines

A vaccine is provided against ZIKY. The vaccine targets Replikin structures in ZIKV thereby limiting replication and virulence. A vaccine may comprise a Replikin peptide or homologue or function fragment of a Replikin peptide identified in a ZIKY. A vaccine may comprise a pharmaceutical composition described herein.

A vaccine may comprise a mixture of a plurality of peptide sequences or homologues of any of SEQ ID NO(s): 1-7, or a mixture of a plurality of peptide sequences and/or homologues of any of SEQ ID NO(s): 1-7. A vaccine may comprise an approximately equal molar mixture of isolated or synthesized peptides of any two or more of SEQ ID NO(s): 1-7, an approximately equal molar mixture of isolated or synthesized peptides of SEQ ID NO(s): 1-7, or an approximately equal molar mixture of the isolated or synthesized peptides of SEQ ID NO(s): 1-7 or homologues thereof.

A pharmaceutical composition or vaccine may comprise a pharmaceutically-acceptable carrier and/or adjuvant and/or excipient. An adjuvant may be a UTOPE. A TUOPE adjuvant may be covalently attached to an isolated or synthesized peptide at the C-terminus, the N-terminus, or both termini. A UTOPE is a peptide sequence of 6 to 10 residues comprising one histidine residue with all other residues being lysine residues.

A non-limiting acceptable carrier, adjuvant, or excipient may include sterile water, oil and water emulsion, keyhole limpet hemocyanin. A non-limiting carrier, excipient, or adjuvant may include a sterile diluent such as water (for dermal, nasal, or ocular application, spraying, or injection), saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. pH can be adjusted with acids or bases, such as hydrochloric acid or sodium hydroxide. Preparations may be enclosed in ampoules, disposable syringes or multiple dose vials made of glass or plastic.

Pharmaceutical compositions suitable for use typically include sterile aqueous solutions (water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In general, a relevant carrier can be a solvent or dispersion medium containing, for example, water, ethanol, polyol (for example, glycerol, propylene glycol, and liquid polyetheylene glycol, and the like), and suitable mixtures thereof.

### Binding Molecules

Binding molecules are provided as an aspect of the invention to target Replikin structures in ZIKY. Replikin peptides may be used to generate antibodies, antibody fragments, or to generate or identify other binding agents, which may be used, for example for diagnostic purposes or to provide passive immunity in an individual. See, e.g., US 2007/0026009 and US 2009/0017052 (each incorporated herein by reference in their entirety).

Various procedures known in the art may be used for the production of antibodies to Replikin sequences or to proteins, protein fragments, polypeptides, or peptides comprising Replikin sequences. Such antibodies include, but are not limited to, polyclonal, monoclonal, chimeric, humanized, single chain, Fab fragments and fragments produced by a Fab expression library. Antibodies that are linked to a cytotoxic agent may also be generated. Antibodies may also be administered in combination with an antiviral agent. Furthermore, combinations of antibodies to different Replikins may be administered as an antibody cocktail.

For the production of antibodies, various host animals or plants may be immunized by injection with a Replikin peptide or a combination of Replikin peptides, including, but not limited to, rabbits, mice, rats, and larger mammals. Monoclonal antibodies to Replikins may be prepared using any technique that provides for the production of antibody molecules. These include but are not limited to the hybridoma technique originally described by Kohler and Milstein, (Nature, 1975, 256:495-497), the human B-cell hybridoma technique (Kosbor et al., 1983, Immunology Today, 4:72), and the EBV hybridoma technique (Cole et al., Monoclonal Antibodies and Cancer Therapy, Alan R. Liss, Inc., pp. 77-96). In addition, techniques developed for the production of chimeric antibodies (Morrison et al., 1984, Proc. Nat. Acad. Sci USA, 81:6851-6855) or other techniques may be used. Alternatively, techniques described for the production of single chain antibodies (U.S. Pat. No. 4,946,778) can be adapted to produce Replikin-specific single chain antibodies. Antibody fragments that contain binding sites for a Replikin may be generated by known techniques. For example, such fragments include but are not limited to F(ab′)2 fragments which can be produced by pepsin digestion of the antibody molecules and the Fab fragments that can be generated by reducing the disulfide bridges of the F(ab′)2 fragments. Alternatively, Fab expression libraries can be generated (Ruse et al., 1989, Science, 246:1275-1281) to allow rapid and easy identification of monoclonal Fab fragments with the desired specificity.

A binding molecule may be any molecule that preferentially binds a Replikin sequence or homologue thereof. A binding molecule may be ligand. A binding molecule may be an antibody or antibody fragment. An antibody fragment may be an F(ab′)2 fragment or Fab fragment or any fragment of an antibody capable of specifically binding a Replikin sequence or homologue thereof. An antibody may be a monoclonal antibody. An antibody may be a humanized antibody. An antibody may be an optimized antibody. In a non-limiting embodiment, the antibody may be an fc-optimized antibody. In a non-limiting embodiment, the antibody is selected using phage display. In a non-limiting embodiment, the antibody is produced from a microorganism such as E. coli.

In a non-limiting embodiment, an antibody, antibody fragment, or binding molecule binds to at least a portion of an amino acid sequence of at least one protein, protein fragment, polypeptide, or peptide comprising a peptide sequence that is 30%, 40%, 50%, 60%, 70%, 80%, 90%, or 95% or more homologous with at least one Replikin peptide sequence identified in ZIKY. An antibody, antibody fragment, or binding agent may be isolated or synthesized. An antibody or antibody fragment may be a monoclonal antibody or monoclonal antibody processed to create an antibody fragment. An antibody or antibody fragment may be made from any biological or chemical method.

### Methods of Predicting Outbreaks of ZIKV

A non-limiting embodiment provides a method of differentiating between relatively more virulent and relatively less virulent forms of ZIKY. A first non-limiting embodiment provides a method of identifying and/or diagnosing a relatively more virulent form of ZIKV comprising determining the Replikin concentration of at least one portion of at least one protein of at least one isolate of ZIKV or at least one portion of at least one gene that expresses at least one protein of the at least one isolate of ZIKV and comparing the Replikin concentration of the at least one isolate of ZIKV to a comparable Replikin concentration in at least one other isolate of ZIKV. In a further non-limiting embodiment, the at least one portion of at least one protein comprises the entirety of at least one protein expressed in a ZIKV and the comparable Replikin concentration is the Replikin concentration of the entirety of the same protein expressed in a ZIKV from the at least one other isolate of the same ZIKY. In a non-limiting embodiment, the Replikin concentration of the at least one isolate of ZIKV is a mean of Replikin concentrations determined in a plurality of isolates of the same ZIKY. In a further non-limiting embodiment, the Replikin concentration of the at least one other isolate of ZIKV is a mean of Replikin concentration determined in a plurality of other isolates of the same ZIKY. In a further non-limiting embodiment, the plurality of isolates of ZIKV is a collection of isolates isolated in a given year and the plurality of other isolates of ZIKV is a collection of isolates of the same ZIKV isolated in a different year. In a further non-limiting embodiment, the Replikin concentration of the more virulent isolate of ZIKV is 3.0 or greater, 4.0 or greater, 5.0 or greater, 6.0 or greater, 8.0 or greater, 10.0 or greater, or 12.0 or greater per 100 amino acid residues. In a further non-limiting embodiment, the Replikin concentration of the more virulent isolate of ZIKV is 8.0 or greater per 100 amino acid residues. In a further non-limiting embodiment, a vaccine is manufactured following the differentiation between relatively more virulent and relatively less virulent forms of ZIKY. In a further non-limiting embodiment, a vaccine is manufactured following prediction of an outbreak of ZIKV following identification of a more virulent form of a ZIKY. In a further non-limiting embodiment, the vaccine comprises at least one structure of the isolate of ZIKB differentiated as relatively more virulent. In a further non-limiting embodiment, the vaccine comprises at least one Replikin peptide sequence identified in the isolate of ZIKB differentiated as relatively virulent.

In a further non-limiting embodiment, the Replikin concentration of the at least one isolate of ZIKV is greater than the Replikin concentration of the at least one other isolate of ZIKY. In a further non-limiting embodiment the Replikin concentration is a mean Replikin concentration of a plurality of isolates with standard deviation from the mean and the standard deviation from the mean is greater than the standard deviation from the mean Replikin concentration of a plurality of other isolates.

Another non-limiting embodiment provides a method of determining an increased probability of an outbreak of ZIKV within about one year following an increase in Replikin concentration in an isolate of ZIKV comprising identifying an increase in the concentration of Replikin sequences in at least one first isolate of a ZIKV as compared to at least one other isolate of the same kind of ZIKV wherein said at least one first isolate is isolated at a later time period than said one other isolate and wherein said increase in the concentration of Replikin sequences signifies the increased probability of the outbreak of the ZIKV within about one year following the increase in the concentration of Replikin sequences.

In a non-limiting embodiment, a method of prediction comprises: (1) obtaining a plurality of isolates of a ZIKV wherein at least one of said isolates is isolated about six months to about 3 years later than at least one other of said isolates; (2) analyzing the amino acid sequence of at least one protein or protein fragment in each isolate of the plurality of isolates for the presence and concentration of Replikin sequences; (3) comparing the concentrations of Replikin sequences in the at least one protein or protein fragment in each isolate of the plurality of isolates one to another; (4) identifying an increase in the concentration of Replikin sequences in said plurality of isolates over at least one time period of about six months or greater; and (5) predicting an outbreak of the ZIKV within about one month to about three years following said identified increase in the concentration of Replikin sequences. In another embodiment of the invention, the outbreak of ZIKV is predicted within about six months. In a further embodiment of the invention, the outbreak of ZIKV virus is predicted within about one year to about three years. In a further non-limiting embodiment, the method of prediction further comprises processing at least one step of the method on a computer.

In a further non-limiting embodiment, the method of prediction further comprises comparison of the standard deviation from the mean of Replikin concentrations of isolates of ZIKV from a given time period, such as a given month, a given year, or any other given time period. In a further non-limiting embodiment, the Replikin concentration is a mean Replikin concentration of a plurality of isolates with standard deviation from the mean and the standard deviation from the mean is greater than the standard deviation from the mean Replikin concentration of a plurality of other isolates.

A further non-limiting embodiment provides a computer readable medium having stored thereon or signal containing instructions which, when executed, cause a processor to perform a method of predicting an expansion of a strain of ZIKV or an increase in virulence or morbidity of ZIKY. In a further embodiment, the processor reports a prediction to a display, user, researcher, or other machine or person. In a further embodiment, the processor identifies to a display, user, researcher, or other machine or person, a portion of a pathogen predicted to be an expanding ZIKV or predicted to increase in virulence or morbidity wherein said portion may be employed as a therapeutic or diagnostic compound. Said portion may be a Replikin peptide or plurality of Replikin peptides or any other structure or portion of said genome of said pathogen including a Replikin Peak Gene. In a non-limiting embodiment, said portion is synthesized and prepared in a pharmaceutical composition.

Another non-limiting embodiment provides a computer system, including a processor coupled to a network and a memory coupled to the processor, the memory containing a plurality of instructions to perform a method of predicting the relative virulence of at least one first group of ZIKV as compared to at least one second group of ZIKY.

Another non-limiting embodiment provides a machine-readable storage medium having stored thereon executable instructions that, when executed by a processor, cause the processor to provide sufficient data to a user, a display, or a printout such that said user or a user of said display or said printout may predict the virulence of a ZIKV based on regression analysis. Another non-limiting embodiment provides a computer system, comprising: a processor coupled to a network; a memory coupled to the processor, the memory containing a plurality of instructions to perform a method of predicting the virulence of an ZIKV based on regression analysis.

### Nucleic Acid Sequences

A non-limiting aspect of the present invention provides a nucleic acid sequence that is antisense to a nucleic acid that encodes for any Replikin peptide present in or identified in a ZIKV isolate. This may include one of SEQ ID NO(s): 1-7 or a small interfering nucleic acid sequence that interferes with a nucleic acid sequence that is 30%, 40%, 50%, 60%, 70%, 80%, 90%, or 95% or more homologous with a nucleic acid that encodes any Replikin peptide of a ZIKV including, for example, anyone of SEQ ID NO(s): 1-7 or is 30%, 40%, 50%, 60%, 70%, 80%, 90%, 95% or more homologous with a nucleic acid that is antisense to a nucleic acid that encodes for anyone of SEQ ID NO(s): 1-7. A nucleic acid sequence may be 21 to 150 nucleotides in length. A nucleic acid sequence may be up to 5, 10, 15, 20, 25, 30, 35, 40, 45, or 50 nucleotides in length.

### Example 1

### Increase in ZIKV Replikin Concentration in May 2015 Predicted the ZIKV Outbreak in Brazil

The Replikins Global Surveillance System™ found a spike increase in ZIKV gene Replikin concentration in May and October of 2015, which correlates with and predicts the outbreak of ZIKV infection and microcephaly cases in Brazil in 2015 and early 2016.

Knowledge of the ZIKV Replikin concentration increase in early 2015 provided more than six months to prepare and to test public health responses and vaccine candidates. The new technology described herein provides real-time analysis that is has allowed for the immediate design of the vaccine and therapeutics described herein that may be immediately tested in response to the ongoing outbreak of ZIKY.

Specific gene sequences in the infectious organism have been found, named Replikins because of their association with rapid replication and population expansion of pathogens. The increasing gene Replikin concentration has been found to predict, one to two years in advance, outbreaks and the course and termination of virus and bacterial pathogenic activity (1) and to provide time to respond in advance of the outbreak.

FIG. 1 and related analyses were generated from data accessed at the PubMed NCBI database website for Zika virus isolates from 1947 through 2015. The information for all accessed individual isolates is provided in Table 1 below including calculated Replikin concentration and year reported for each sequence.

TABLE 1
Replikin concentration data determined from sequences available from PubMed
NCBI database for individual reported isolates from 1947 through 2015

| | REPLIKIN | | | | | |
|---|---|---|---|---|---|---|
| ACCESSION | CONCEN- | | | | | |
| NO | TRATION | YEAR | SOURCE | SEROTYPE | STRAIN | DEFINITION |
| AEN75263 | 2.9 | 1947 | Zikavirus | unknown | MR 766 | Direct Submission Genetic characterization of |
| | | | | | | zika vims strains: geographic expansion of the |
| | | | | | | asian lineage |
| AHL43457 | 8.6 | 1963 | Zikavirus | unknown | MR1429 | Molecular evolution of Zika vims during its |
| | | | | | | emergence in the 20(th) century Direct |
| | | | | | | Submission |
| AEN75264 | 2.8 | 1966 | Zikavirus | unknown | P6-740 | Direct Submission Genetic characterization of |
| | | | | | | zika vims strains: geographic expansion of the |
| | | | | | | asian lineage |
| AEN75265 | 2.9 | 1968 | Zikavirus | unknown | IbH 30656 | Direct Submission Genetic characterization of |
| | | | | | | zika vims strains: geographic expansion of the |
| | | | | | | asian lineage |
| AHL43470 | 3.4 | 1969 | Zikavirus | unknown | ArD9957 | Molecular evolution of Zika vims during its |
| | | | | | | emergence in the 20(th) century Direct |
| | | | | | | Submission |
| AHL43453 | 9.6 | 1969 | Zikavirus | unknown | ArD9957 | Molecular evolution of Zika vims during its |
| | | | | | | emergence in the 20(th) century Direct |
| | | | | | | Submission |
| AHF49783 | 2.9 | 1976 | Zikavirus | unknown | ARB 13565 | Direct Submission Molecular characterization |
| | | | | | | of three Zika flavivimses obtained from |
| | | | | | | sylvatic mosquitoes in the Central African |
| | | | | | | Republic |
| AHL43499 | 3.4 | 1979 | Zikavirus | unknown | AnD30332 | Molecular evolution of Zika vims during its |
| | | | | | | emergence in the 20(th) century Direct |
| | | | | | | Submission |
| AHL43473 | 4.2 | 1979 | Zikavirus | unknown | ArD30101 | Molecular evolution of Zika vims during its |
| | | | | | | emergence in the 20(th) century Direct |
| | | | | | | Submission |
| AHL43472 | 3.4 | 1979 | Zikavirus | unknown | ArD30l56 | Molecular evolution of Zika vims during its |
| | | | | | | emergence in the 20(th) century Direct |
| | | | | | | Submission |
| AHL43456 | 9.6 | 1979 | Zikavirus | unknown | ArD30l56 | Molecular evolution of Zika vims during its |
| | | | | | | emergence in the 20(th) century Direct |
| | | | | | | Submission |
| AHL43455 | 9.6 | 1979 | Zikavirus | unknown | AnD30332 | Molecular evolution of Zika vims during its |
| | | | | | | emergence in the 20(th) century Direct |
| | | | | | | Submission |
| AHL43454 | 9.6 | 1979 | Zikavirus | unknown | ArD30101 | Molecular evolution of Zika vims during its |
| | | | | | | emergence in the 20(th) century Direct |
| | | | | | | Submission |
| AHL43498 | 3.4 | 1980 | Zikavirus | unknown | ArA1465 | Molecular evolution of Zika vims during its |
| | | | | | | emergence in the 20(th) century Direct |
| | | | | | | Submission |
| AHL43442 | 9.6 | 1980 | Zikavirus | unknown | ArA1465 | Molecular evolution of Zika vims during its |
| | | | | | | emergence in the 20(th) century Direct |
| | | | | | | Submission |
| AHL43494 | 3.4 | 1981 | Zikavirus | unknown | ArAn18 | Molecular evolution of Zika vims during its |
| | | | | | | emergence in the 20(th) century Direct |
| | | | | | | Submission |
| AHL43452 | 9.6 | 1981 | Zikavirus | unknown | ArAn18 | Molecular evolution of Zika vims during its |
| | | | | | | emergence in the 20(th) century Direct |
| | | | | | | Submission |
| AEN75266 | 2.7 | 1984 | Zikavirus | unknown | ArD 41519 | Direct Submission Genetic characterization |
| | | | | | | of zika virus strains: geographic expansion of |
| | | | | | | the asian lineage |
| AHL43497 | 3.4 | 1990 | Zikavirus | unknown | ArA27096 | Molecular evolution of Zika virus during its |
| | | | | | | emergence in the 20(th) century Direct |
| | | | | | | Submission |
| AHL43496 | 3.4 | 1990 | Zikavirus | unknown | ArA27 10 1 | Molecular evolution of Zika virus during its |
| | | | | | | emergence in the 20(th) century Direct |
| | | | | | | Submission |
| AHL43495 | 3.4 | 1990 | Zikavirus | unknown | ArA27 106 | Molecular evolution of Zika virus during its |
| | | | | | | emergence in the 20(th) century Direct |
| | | | | | | Submission |
| AHL43493 | 3.4 | 1990 | Zikavirus | unknown | ArA27290 | Molecular evolution of Zika virus during its |
| | | | | | | emergence in the 20(th) century Direct |
| | | | | | | Submission |
| AHL43492 | 3.4 | 1990 | Zikavirus | unknown | ArA27407 | Molecular evolution of Zika virus during its |
| | | | | | | emergence in the 20(th) century Direct |
| | | | | | | Submission |
| AHL43491 | 3.4 | 1990 | Zikavirus | unknown | ArA27443 | Molecular evolution of Zika virus during its |
| | | | | | | emergence in the 20(th) century Direct |
| | | | | | | Submission |
| AHL43467 | 9.6 | 1990 | Zikavirus | unknown | ArA27 106 | Molecular evolution of Zika virus during its |
| | | | | | | emergence in the 20(th) century Direct |
| | | | | | | Submission |
| AHL43466 | 9.6 | 1990 | Zikavirus | unknown | ArA27407 | Molecular evolution of Zika virus during its |
| | | | | | | emergence in the 20(th) century Direct |
| | | | | | | Submission |
| AHL43465 | 9.6 | 1990 | Zikavirus | unknown | ArA27443 | Molecular evolution of Zika virus during its |
| | | | | | | emergence in the 20(th) century Direct |
| | | | | | | Submission |
| AHL43464 | 7.2 | 1990 | Zikavirus | unknown | ArA27096 | Molecular evolution of Zika virus during its |
| | | | | | | emergence in the 20(th) century Direct |
| | | | | | | Submission |
| AHL43463 | 9.6 | 1990 | Zikavirus | unknown | ArA27290 | Molecular evolution of Zika virus during its |
| | | | | | | emergence in the 20(th) century Direct |
| | | | | | | Submission |
| AHL43462 | 5.6 | 1990 | Zikavirus | unknown | ArA27 10 1 | Molecular evolution of Zika virus during its |
| | | | | | | emergence in the 20(th) century Direct |
| | | | | | | Submission |
| AHL43469 | 5.1 | 1991 | Zikavirus | unknown | HD78788 | Molecular evolution of Zika virus during its |
| | | | | | | emergence in the 20(th) century Direct |
| | | | | | | Submission |
| AHL43461 | 9.6 | 1991 | Zikavirus | unknown | HD78788 | Molecular evolution of Zika virus during its |
| | | | | | | emergence in the 20(th) century Direct |
| | | | | | | Submission |
| AHL43490 | 3.4 | 1996 | Zikavirus | unknown | ArA506 | Molecular evolution of Zika virus during its |
| | | | | | | emergence in the 20(th) century Direct |
| | | | | | | Submission |
| AHL43459 | 9.6 | 1996 | Zikavirus | unknown | ArA506 | Molecular evolution of Zika virus during its |
| | | | | | | emergence in the 20(th) century Direct |
| | | | | | | Submission |
| AHL43487 | 3.4 | 1997 | Zikavirus | unknown | ArD127707 | Molecular evolution of Zika virus during its |
| | | | | | | emergence in the 20(th) century Direct |
| | | | | | | Submission |
| AHL43486 | 3.4 | 1997 | Zikavirus | unknown | ArD 127710 | Molecular evolution of Zika virus during its |
| | | | | | | emergence in the 20(th) century Direct |
| | | | | | | Submission |
| AHL43485 | 3.4 | 1997 | Zikavirus | unknown | ArDI27984 | Molecular evolution of Zika virus during its |
| | | | | | | emergence in the 20(th) century Direct |
| | | | | | | Submission |
| AHL43484 | 3.4 | 1997 | Zikavirus | unknown | ArDI27987 | Molecular evolution of Zika virus during its |
| | | | | | | emergence in the 20(th) century Direct |
| | | | | | | Submission |
| AHL43483 | 3.4 | 1997 | Zikavirus | unknown | ArDI27988 | Molecular evolution of Zika virus during its |
| | | | | | | emergence in the 20(th) century Direct |
| | | | | | | Submission |
| AHL43482 | 3.4 | 1997 | Zikavirus | unknown | ArDI27994 | Molecular evolution of Zika virus during |
| | | | | | | its emergence in the 20(th) centnry Direct |
| | | | | | | Submission |
| AHL43449 | 9.6 | 1997 | Zikavirus | unknown | ArD 127710 | Molecular evolution of Zika vims during its |
| | | | | | | emergence in the 20(th) centnry Direct |
| | | | | | | Submission |
| AHL43448 | 9.6 | 1997 | Zikavirus | unknown | ArDI27994 | Molecular evolution of Zika vims during its |
| | | | | | | emergence in the 20(th) centnry Direct |
| | | | | | | Submission |
| AHL43447 | 9.6 | 1997 | Zikavirus | unknown | ArD127707 | Molecular evolution of Zika vims during its |
| | | | | | | emergence in the 20(th) centnry Direct |
| | | | | | | Submission |
| AHL43446 | 9.6 | 1997 | Zikavirus | unknown | ArDI27984 | Molecular evolution of Zika vims during its |
| | | | | | | emergence in the 20(th) centnry Direct |
| | | | | | | Submission |
| AHL43445 | 9.6 | 1997 | Zikavirus | unknown | ArDI27987 | Molecular evolution of Zika vims during its |
| | | | | | | emergence in the 20(th) centnry Direct |
| | | | | | | Submission |
| AHL43444 | 9.6 | 1997 | Zikavirus | unknown | ArDI27988 | Molecular evolution of Zika vims during its |
| | | | | | | emergence in the 20(th) centnry Direct |
| | | | | | | Submission |
| AAC58803 | 3.2 | 1997 | Zikavirus | unknown | MR-766 | Phylogeny of the genus Flavivims Direct |
| | | | | | | Submission |
| AHL43481 | 3 | 1998 | Zikavirus | unknown | ArD132912 | Molecular evolution of Zika vims during its |
| | | | | | | emergence in the 20(th) centnry Direct |
| | | | | | | Submission |
| AHL43480 | 5.1 | 1998 | Zikavirus | unknown | ArD132915 | Molecular evolution of Zika vims during its |
| | | | | | | emergence in the 20(th) centnry Direct |
| | | | | | | Submission |
| AHL43443 | 9.6 | 1998 | Zikavirus | unknown | ArD132912 | Molecular evolution of Zika vims during its |
| | | | | | | emergence in the 20(th) centnry Direct |
| | | | | | | Submission |
| AHL43441 | 9.6 | 1998 | Zikavirus | unknown | ArD132915 | Molecular evolution of Zika vims during its |
| | | | | | | emergence in the 20(th) centnry Direct |
| | | | | | | Submission |
| NP 003712 | 0.8 | 1999 | Homo | unknown | human | The ankyrin repeat-containing adaptor protein |
| | | | sapiens | | | Tvl-1 is a novel substrate and regulator ofRaf1 |
| | | | (human) | | | The 752delG26 mutation in the RFXANK gene |
| | | | | | | associated with major histocompatibility |
| | | | | | | complex class II deficiency: evidence for a |
| | | | | | | founder effect in the Moroccan population |
| | | | | | | Founder effect for a 26-bp deletion in the |
| | | | | | | RFXANK gene in North African major |
| | | | | | | histocompatibility complex class II-deficient |
| | | | | | | patients belonging to complementation group B |
| | | | | | | Maj or histocompatibility complex class II |
| | | | | | | expression deficiency caused by a RFXANK |
| | | | | | | founder mutation: a survey of 35 patients |
| | | | | | | Novel mutations within the RFX-B gene and |
| | | | | | | partial rescue of MHC and related genes |
| | | | | | | through exogenous class II transactivator in |
| | | | | | | RFX-Bdeficient cells Identification of the |
| | | | | | | ankyrin repeat proteins ANKRA and RFXANK |
| | | | | | | as novel partners of class IIa histone |
| | | | | | | deacetylases New functions of the major |
| | | | | | | histocompatibility complex class II-specific |
| | | | | | | transcription factor RFXANK revealed by a |
| | | | | | | high-resolution mutagenesis stndy RFX-B is |
| | | | | | | the gene responsible for the most common |
| | | | | | | cause of the bare lymphocyte syndrome; an |
| | | | | | | MHC class II immunodeficiency A gene |
| | | | | | | encoding a novel RFX-associated |
| | | | | | | transactivator is mutated in the majority of |
| | | | | | | MHC class II deficiency patients Assembly of |
| | | | | | | the RFX complex on the MHCII promoter: role |
| | | | | | | of RFXAP and RFXB in relieving |
| | | | | | | autoinhibition of RFX5 |
| AHL43489 | 3.4 | 1999 | Zikavirus | unknown | ArA982 | Molecular evolution of Zika vims during |
| | | | | | | its emergence in the 20(th) centnry Direct |
| | | | | | | Submission |
| AHL43488 | 3.4 | 1999 | Zikavirus | unknown | ArA986 | Molecular evolution of Zika vims during |
| | | | | | | its emergence in the 20(th) century |
| | | | | | | Direct Submission |
| AHL43471 | 3.4 | 1999 | Zikavirus | unknown | ArA975 | Molecular evolution of Zika vims during |
| | | | | | | its emergence in the 20(th) century |
| | | | | | | Direct Submission |
| AHL43468 | 9.6 | 1999 | Zikavirus | unknown | ArA982 | Molecular evolution of Zika vims during |
| | | | | | | its emergence in the 20(th) century |
| | | | | | | Direct Submission |
| AHL43460 | 9.6 | 1999 | Zikavirus | unknown | ArA986 | Molecular evolution of Zika vims during |
| | | | | | | its emergence in the 20(th) century |
| | | | | | | Direct Submission |
| AHL43458 | 9.6 | 1999 | Zikavirus | unknown | ArA975 | Molecular evolution of Zika vims during |
| | | | | | | its emergence in the 20(th) century |
| | | | | | | Direct Submission |
| AHL43479 | 3 | 2000 | Zikavirus | unknown | ArD141170 | Molecular evolution of Zika vims during |
| | | | | | | its emergence in the 20(th) century |
| | | | | | | Direct Submission |
| AHL43440 | 9.6 | 2000 | Zikavirus | unknown | ArD141170 | Molecular evolution of Zika vims during |
| | | | | | | its emergence in the 20(th) century |
| | | | | | | Direct Submission |
| AHL43478 | 3 | 2001 | Zikavirus | unknown | ArDl49810 | Molecular evolution of Zika vims during |
| | | | | | | its emergence in the 20(th) century |
| | | | | | | Direct Submission |
| AHL43477 | 3 | 2001 | Zikavirus | unknown | ArDl47917 | Molecular evolution of Zika vims during |
| | | | | | | its emergence in the 20(th) century |
| | | | | | | Direct Submission |
| AHL43476 | 3 | 2001 | Zikavirus | unknown | ArDl49938 | Molecular evolution of Zika vims during |
| | | | | | | its emergence in the 20(th) century |
| | | | | | | Direct Submission |
| AHL43439 | 9.6 | 2001 | Zikavirus | unknown | ArDl49938 | Molecular evolution of Zika vims during |
| | | | | | | its emergence in the 20(th) century |
| | | | | | | Direct Submission |
| AHL43438 | 9.2 | 2001 | Zikavirus | unknown | ArDl47917 | Molecular evolution of Zika vims during |
| | | | | | | its emergence in the 20(th) century |
| | | | | | | Direct Submission |
| AHL43437 | 9.6 | 2001 | Zikavirus | unknown | ArDl49810 | Molecular evolution of Zika vims during |
| | | | | | | its emergence in the 20(th) century |
| | | | | | | Direct Submission |
| AAK91609 | 7.6 | 2001 | Zikavirus | unknown | unknown | Phylogenetic relationships of flavivimses |
| | | | | | | correlate with their epidemiology; disease |
| | | | | | | association and biogeography Direct Sumission |
| AHL43475 | 3.4 | 2002 | Zikavirus | unknown | ArDl65522 | Molecular evolution of Zika vims during |
| | | | | | | its emergence in the 20(th) century |
| | | | | | | Direct Submission |
| AHL43474 | 3.4 | 2002 | Zikavirus | unknown | ArDl65531 | Molecular evolution of Zika vims during |
| | | | | | | its emergence in the 20(th) century |
| | | | | | | Direct Submission |
| AHL43451 | 9.6 | 2002 | Zikavirus | unknown | ArDl65522 | Molecular evolution of Zika vims during its |
| AHL43450 | 9.6 | 2002 | Zikavirus | unknown | ArDl65531 | Molecular evolution of Zika vims during |
| | | | | | | its emergence in the 20(th) century |
| | | | | | | Direct Submission |
| AHLl6749 | 8.6 | 2007 | Zikavirus | unknown | CCB-870 | Zika Vims in Gabon (Central Africa) -2007: |
| | | | | | | A New Threat from Aedes albopictus? Direct |
| AAV34161 | 2.8 | 2005 | Bagaza | unknown | DakAr B209 | Full-length sequencing and genomic |
| | | | vims | | | characterization of Bagaza; Kedougou; and |
| | | | | | | Zika vimses Biological transmission of |
| | | | | | | arbovimses: reexamination of and new insights |
| | | | | | | into components; mechanisms; and unique |
| | | | | | | traits as well as their evolutionary trends Direct |
| | | | | | | Submission Direct Submission |
| AHLl6750 | 1.2 | 2007 | Zikavirus | unknown | CCB-870 | Zika Vims in Gabon (Central Africa) -2007: A |
| | | | | | | New Threat from Aedes albopictus? Direct |
| | | | | | | Submission |
| AAV34151 | 2.8 | 2005 | Zikavirus | unknown | MR 766 | Full-length sequencing and genomic |
| | | | | | | characterization of Bagaza; Kedougou; and |
| | | | | | | Zika vimses Biological transmission of |
| | | | | | | arbovimses: reexamination of and new insights |
| | | | | | | into components; mechanisms; and unique |
| | | | | | | traits as well as their evolutionary trends Direct |
| | | | | | | Submission Direct Submission |
| AAV34156 | 2.9 | 2005 | Kedougou | unknown | DakAar Dl470 | Full-length sequencing and genomic |
| | | | vims | | | characterization of Bagaza; Kedougou; and |
| | | | | | | Zika vimses Biological transmission of |
| | | | | | | arbovimses: reexamination of and new insights |
| | | | | | | into components; mechanisms; and unique |
| | | | | | | traits as well as their evolutionary trends Direct |
| | | | | | | Submission Direct Submission |
| ACD75819 | 3 | 2007 | Zikavirus | unknown | unknown | Direct Submission Genetic and serologic |
| | | | | | | properties of Zika vims associated with an |
| | | | | | | epidemic; Yap State; Micronesia; 2007 |
| YP 002790883 | 2.8 | 2009 | Bagaza virus | unknown | DakAr B209 | Full-length sequencing and genomic |
| | | | | | | characterization of Bagaza; Kedougou; and |
| | | | | | | Zika vimses Biological transmission of |
| | | | | | | arbovimses: reexamination of and new insights |
| | | | | | | into components; mechanisms; and unique |
| | | | | | | traits as well as their evolutionary trends Direct |
| | | | | | | Submission Direct Submission Direct |
| | | | | | | Submission |
| YP 002790881 | 2.8 | 2009 | Zikavirus | unknown | MR 766 | Full-length sequencing and genomic |
| | | | | | | characterization of Bagaza; Kedougou; and |
| | | | | | | Zika vimses Biological transmission of |
| | | | | | | arbovimses: reexamination of and new insights |
| | | | | | | into components; mechanisms; and unique |
| | | | | | | traits as well as their evolutionary trends Direct |
| | | | | | | Submission Direct Submission Direct |
| | | | | | | Submission |
| YP 002790882 | 2.9 | 2009 | Kedougou virus | unknown | DakAar Dl470 | Full-length sequencing and genomic |
| | | | | | | characterization of Bagaza; Kedougou; and |
| | | | | | | Zika vimses Biological transmission of |
| | | | | | | arbovimses: reexamination of and new insights |
| | | | | | | into components; mechanisms; and unique |
| | | | | | | traits as well as their evolutionary trends Direct |
| | | | | | | Submission Direct Submission Direct |
| | | | | | | Submission |
| ABI54475 | 2.9 | 2009 | Zikavirus | unknown | MR 766 | Direct Submission Genomics and evolution of |
| | | | | | | Aedes-borne flavivimses |
| AHL37808 | 3 | 2013 | Zikavirus | unknown | PLCal ZV | Direct Submission First case of zika vims |
| | | | | | | infection in a returning canadian traveler |
| AHZ08798 | 3 | 2013 | Zikavirus | unknown | Tahiti | Zika vims infection after travel to Tahiti; |
| | | | | | | December 2013 Direct Submission |
| AHZ13508 | 3 | 2013 | Zikavirus | unknown | HIPFI2013 | Direct Submission Complete coding sequence |
| | | | | | | of zika vims from a French polynesia outbreak |
| | | | | | | in 2013 |
| AFD30972 | 3 | 2010 | Zikavirus | unknown | FSS13025 | Direct Submission Genetic characterization of |
| | | | | | | zika vims strains: geographic expansion of the |
| | | | | | | asian lineage |
| ABY86749 | 3.1 | 2008 | Zikavirus | unknown | unknown | Universal primers that amplify RNA from all |
| | | | | | | three flavivims subgroups Direct Submission |
| ABW77724 | 3.9 | 2008 | Zikavirus | unknown | MR 766 | A real-time RT-PCR method for the universal |
| | | | | | | detection and identification of flavivimses |
| | | | | | | Direct Submission |
| AKH87424 | 4.9 | 2012 | Zikavirus | unknown | CPC-0740 | Zika vims infection; Philippines; 2012 Direct |
| | | | | | | Submission |
| AGS07298 | 5.2 | 2012 | Zikavirus | unknown | Java | Zika vims infection acquired during brief travel |
| | | | | | | to indonesia Direct Submission |
| AHF49785 | 2.9 | 2014 | Zikavirus | unknown | ARE770 1 | Direct Submission Molecular characterization of |
| | | | | | | three Zika flavivimses obtained from sylvatic |
| | | | | | | mosquitoes in the Central African Republic |
| AJD8l421 | 1.4 | 2014 | Zikavirus | unknown | NC14170420144554 | Co-infection Zika and Dengue vims in New |
| | | | | | | Caledonia 2014 Direct Submission |
| AHL43501 | 2.7 | 2014 | Zikavirus | unknown | ArD7l17 | Direct Submission Molecular evolution of Zika |
| | | | | | | vims during its emergence in the 20(th) century |
| AHL43502 | 2.8 | 2014 | Zikavirus | unknown | ArD128000 | Direct Submission Molecular evolution of Zika |
| | | | | | | vims during its emergence in the 20(th) century |
| AHL43500 | 2.8 | 2014 | Zikavirus | unknown | ArB 1362 | Direct Submission Molecular evolution of Zika |
| | | | | | | vims during its emergence in the 20(th) century |
| AHF49784 | 2.9 | 2014 | Zikavirus | unknown | ARE 15076 | Direct Submission Molecular characterization of |
| | | | | | | three Zika flavivimses obtained from sylvatic |
| | | | | | | mosquitoes in the Central African Republic |
| BAP47441 | 2.9 | 2014 | Zikavirus | unknown | MR766NIID | Direct Submission Complete nucleotide sequence |
| | | | | | | of Zika vims MR766-NIID strain |
| AHL43505 | 2.9 | 2014 | Zikavirus | unknown | ArD158095 | Direct Submission Molecular evolution of Zika |
| | | | | | | vims during its emergence in the 20(th) century |
| AHL43504 | 2.9 | 2014 | Zikavirus | unknown | ArD158084 | Direct Submission Molecular evolution of Zika |
| | | | | | | vims during its emergence in the 20(th) century |
| AHL43503 | 2.9 | 2014 | Zikavirus | unknown | ArD157995 | Direct Submission Molecular evolution of Zika |
| | | | | | | vims during its emergence in the 20(th) century |
| AJD79051 | 4 | 2014 | Zikavirus | unknown | CHI290901 4 | A report on the outbreak of Zika vims on Easter |
| | | | | | | Island; South Pacific; 2014 Direct Submission |
| AJD79050 | 4 | 2014 | Zikavirus | unknown | CHI290891 4 | A report on the outbreak of Zika vims on Easter |
| | | | | | | Island; South Pacific; 2014 Direct Submission |
| AJD79049 | 4 | 2014 | Zikavirus | unknown | CHI290871 4 | A report on the outbreak of Zika vims on Easter |
| | | | | | | Island; South Pacific; 2014 Direct Submission |
| AJD79048 | 4 | 2014 | Zikavirus | unknown | CHI28710l 4 | A report on the outbreak of Zika vims on Easter |
| | | | | | | Island; South Pacific; 2014 Direct Submission |
| AJD79047 | 4 | 2014 | Zikavirus | unknown | CHI275961 4 | A report on the outbreak of Zika vims on Easter |
| | | | | | | Island; South Pacific; 2014 Direct Submission |
| AJD79046 | 4 | 2014 | Zikavirus | unknown | CHI275951 4 | A report on the outbreak of Zika vims on Easter |
| | | | | | | Island; South Pacific; 2014 Direct Submission |
| AJD79045 | 4 | 2014 | Zikavirus | unknown | CHI275901 4 | A report on the outbreak of Zika vims on Easter |
| | | | | | | Island; South Pacific; 2014 Direct Submission |
| AJD79044 | 4 | 2014 | Zikavirus | unknown | CHI275851 4 | A report on the outbreak of Zika vims on Easter |
| | | | | | | Island; South Pacific; 2014 Direct Submission |
| AJD79043 | 4 | 2014 | Zikavirus | unknown | CHI261301 4 | A report on the outbreak of Zika vims on Easter |
| | | | | | | Island; South Pacific; 2014 Direct Submission |
| AJD79042 | 4 | 2014 | Zikavirus | unknown | CHI249041 4 | A report on the outbreak of Zika vims on Easter |
| | | | | | | Island; South Pacific; 2014 Direct Submission |
| AJD79041 | 4 | 2014 | Zikavirus | unknown | CHI248831 4 | A report on the outbreak of Zika vims on Easter |
| | | | | | | Island; South Pacific; 2014 Direct Submission |
| AJD79040 | 4 | 2014 | Zikavirus | unknown | CHI248821 4 | A report on the outbreak of Zika vims on Easter |
| | | | | | | Island; South Pacific; 2014 Direct Submission |
| AJD79039 | 4 | 2014 | Zikavirus | unknown | CHI248771 4 | A report on the outbreak of Zika vims on Easter |
| | | | | | | Island; South Pacific; 2014 Direct Submission |
| AJD79038 | 4 | 2014 | Zikavirus | unknown | CHI234801 4 | A report on the outbreak of Zika virus on Easter |
| | | | | | | Island; South Pacific; 2014 Direct Submission |
| AJD79037 | 4 | 2014 | Zikavirus | unknown | CHI275921 4 | A report on the outbreak of Zika virus on Easter |
| | | | | | | Island; South Pacific; 2014 Direct Submission |
| AJD79036 | 4 | 2014 | Zikavirus | unknown | CHI275891 4 | A report on the outbreak of Zika virus on Easter |
| | | | | | | Island; South Pacific; 2014 Direct Submission |
| AJD79035 | 4 | 2014 | Zikavirus | unknown | CHI275861 4 | A report on the outbreak of Zika virus on Easter |
| | | | | | | Island; South Pacific; 2014 Direct Submission |
| AJD79034 | 4 | 2014 | Zikavirus | unknown | CHI26 127 1 4 | A report on the outbreak of Zika virus on Easter |
| | | | | | | Island; South Pacific; 2014 Direct Submission |
| AJD79033 | 4 | 2014 | Zikavirus | unknown | CHI261211 4 | A report on the outbreak of Zika virus on Easter |
| | | | | | | Island; South Pacific; 2014 Direct Submission |
| AJD79032 | 4 | 2014 | Zikavirus | unknown | CHI261171 4 | A report on the outbreak of Zika virus on Easter |
| | | | | | | Island; South Pacific; 2014 Direct Submission |
| AJD79031 | 4 | 2014 | Zikavirus | unknown | CHI261151 4 | A report on the outbreak of Zika virus on Easter |
| | | | | | | Island; South Pacific; 2014 Direct Submission |
| AJD79030 | 4 | 2014 | Zikavirus | unknown | CHI261121 4 | A report on the outbreak of Zika virus on Easter |
| | | | | | | Island; South Pacific; 2014 Direct Submission |
| AJD79029 | 4 | 2014 | Zikavirus | unknown | CHI248981 4 | A report on the outbreak of Zika virus on Easter |
| | | | | | | Island; South Pacific; 2014 Direct Submission |
| AJD79028 | 4 | 2014 | Zikavirus | unknown | CHI248781 4 | A report on the outbreak of Zika virus on Easter |
| | | | | | | Island; South Pacific; 2014 Direct Submission |
| AJD79027 | 4 | 2014 | Zikavirus | unknown | CHI248741 4 | A report on the outbreak of Zika virus on Easter |
| | | | | | | Island; South Pacific; 2014 Direct Submission |
| AJD79026 | 4 | 2014 | Zikavirus | unknown | CHI248731 4 | A report on the outbreak of Zika virus on Easter |
| | | | | | | Island; South Pacific; 2014 Direct Submission |
| AJD79025 | 4 | 2014 | Zikavirus | unknown | CHI234781 4 | A report on the outbreak of Zika virus on Easter |
| | | | | | | Island; South Pacific; 2014 Direct Submission |
| AJD79024 | 4 | 2014 | Zikavirus | unknown | CHI234761 4 | A report on the outbreak of Zika virus on Easter |
| | | | | | | Island; South Pacific; 2014 Direct Submission |
| AJD79023 | 4 | 2014 | Zikavirus | unknown | CHI234741 4 | A report on the outbreak of Zika virus on Easter |
| | | | | | | Island; South Pacific; 2014 Direct Submission |
| AJD79022 | 4 | 2014 | Zikavirus | unknown | CHI234721 4 | A report on the outbreak of Zika virus on Easter |
| | | | | | | Island; South Pacific; 2014 Direct Submission |
| AJD79021 | 4 | 2014 | Zikavirus | unknown | CHI228391 4 | A report on the outbreak of Zika virus on Easter |
| | | | | | | Island; South Pacific; 2014 Direct Submission |
| AJD79020 | 4 | 2014 | Zikavirus | unknown | CHI228381 4 | A report on the outbreak of Zika virus on Easter |
| | | | | | | Island; South Pacific; 2014 Direct Submission |
| AJD79019 | 4 | 2014 | Zikavirus | unknown | CHI228351 4 | A report on the outbreak of Zika virus on Easter |
| | | | | | | Island; South Pacific; 2014 Direct Submission |
| AJD79018 | 4 | 2014 | Zikavirus | unknown | CHI228301 4 | A report on the outbreak of Zika virus on Easter |
| | | | | | | Island; South Pacific; 2014 Direct Submission |
| AJD79017 | 4 | 2014 | Zikavirus | unknown | CHI209141 4 | A report on the outbreak of Zika virus on Easter |
| | | | | | | Island; South Pacific; 2014 Direct Submission |
| AJD79016 | 4 | 2014 | Zikavirus | unknown | CHI209111 4 | A report on the outbreak of Zika virus on Easter |
| | | | | | | Island; South Pacific; 2014 Direct Submission |
| AJD79015 | 4 | 2014 | Zikavirus | unknown | CHI228281 4 | A report on the outbreak of Zika virus on Easter |
| | | | | | | Island; South Pacific; 2014 Direct Submission |
| AJD79014 | 4 | 2014 | Zikavirus | unknown | CHIl80511 4 | A report on the outbreak of Zika virus on Easter |
| | | | | | | Island; South Pacific; 2014 Direct Submission |
| AJD79013 | 4 | 2014 | Zikavirus | unknown | CHIl80501 4 | A report on the outbreak of Zika virus on Easter |
| | | | | | | Island; South Pacific; 2014 Direct Submission |
| AJD79012 | 4 | 2014 | Zikavirus | unknown | CHIl75771 4 | A report on the outbreak of Zika virus on Easter |
| | | | | | | Island; South Pacific; 2014 Direct Submission |
| AJD79011 | 4 | 2014 | Zikavirus | unknown | CHIl80531 4 | A report on the outbreak of Zika virus on Easter |
| | | | | | | Island; South Pacific; 2014 Direct Submission |
| AJD79010 | 4 | 2014 | Zikavirus | unknown | CHIl47281 4 | A report on the outbreak of Zika virus on Easter |
| | | | | | | Island; South Pacific; 2014 Direct Submission |
| AJD79009 | 4 | 2014 | Zikavirus | unknown | CHIl41041 4 | A report on the outbreak of Zika virus on Easter |
| | | | | | | Island; South Pacific; 2014 Direct Submission |
| AJD79008 | 4 | 2014 | Zikavirus | unknown | CHIl41021 4 | A report on the outbreak of Zika virus on Easter |
| | | | | | | Island; South Pacific; 2014 Direct Submission |
| AJD79007 | 4 | 2014 | Zikavirus | unknown | CHIl37961 4 | A report on the outbreak of Zika virus on Easter |
| | | | | | | Island; South Pacific; 2014 Direct Submission |
| AJD79006 | 4 | 2014 | Zikavirus | unknown | CHIl37951 4 | A report on the outbreak of Zika virus on Easter |
| | | | | | | Island; South Pacific; 2014 Direct Submission |
| AJD79005 | 4 | 2014 | Zikavirus | unknown | CHIl05851 4 | A report on the outbreak of Zika virus on Easter |
| | | | | | | Island; South Pacific; 2014 Direct Submission |
| AJD79004 | 4 | 2014 | Zikavirus | unknown | CHIl13121 4 | A report on the outbreak of Zika virus on Easter |
| | | | | | | Island; South Pacific; 2014 Direct Submission |
| AJD79003 | 4 | 2014 | Zikavirus | unknown | CHI290881 4 | A report on the outbreak of Zika virus on Easter |
| | | | | | | Island; South Pacific; 2014 Direct Submission |
| AJD79002 | 4 | 2014 | Zikavirus | unknown | CHI228371 4 | A report on the outbreak of Zika virus on Easter |
| | | | | | | Island; South Pacific; 2014 Direct Submission |
| AJD79001 | 4 | 2014 | Zikavirus | unknown | CHIl80521 4 | A report on the outbreak of Zika virus on Easter |
| | | | | | | Island; South Pacific; 2014 Direct Submission |
| AJA40023 | 4.4 | 2014 | Zikavirus | unknown | NCI4-03042014-3481 | Detection of zika virus in urine Direct |
| | | | | | | Submission |
| AJD81422 | 4.6 | 2014 | virus | unknown | NCI4-23012014-250 | Co-infection Zika and Dengue virus in in |
| | | | | | | New Caledonia 2014 Direct Submission |
| AKH87423 | 4.9 | 2014 | Zikavirus | unknown | SVOI27!l4 | Detection of Zika Virus Infection in Thailand; |
| | | | | | | 2012-2014 Direct Submission |
| AJA40024 | 4.9 | 2014 | Zikavirus | unknown | NCI4-02042014-3220 | Detection of zika virus in urine |
| | | | | | | Direct Submission |
| AIC06934 | 6.7 | 2014 | Zikavirus | unknown | CK-ISL 2014 | Imported Zika virus infection from the Cook |
| | | | | | | Islands into Australia; 2014 Direct Submission |
| AJD81420 | 6.9 | 2014 | virus | unknown | NCI4-17042014-4554 | Co-infection Zika and Dengue virus in |
| | | | | | | New Caledonia 2014 Direct Submission |
| ALY05362 | 2.6 | 2015 | Zikavirus | unknown | BRl949/15 | Direct Submission |
| ALX35659 | 3 | 2015 | Zikavirus | unknown | Zl106033 | Zika vims genome from the Americas Direct |
| | | | | | | Submission |
| ALU33341 | 3 | 2015 | Zikavirus | unknown | ZikaSPH20 15 | Direct Submission |
| ALX35661 | 4.1 | 2015 | Zikavirus | unknown | Zl106031 | Zika vims genome from the Americas Direct |
| | | | | | | Submission |
| ALX35660 | 4.1 | 2015 | Zikavirus | unknown | Zl106032 | Zika vims genome from the Americas Direct |
| | | | | | | Submission |
| ALX35662 | 5.5 | 2015 | Zikavirus | unknown | Zl106027 | Zika vims genome from the Americas Direct |
| | | | | | | Submission |
| AL157106 | 10.7 | 2015 | Zikavirus | BR | BRlUFBN | Zika Vims Outbreak; Bahia; Brazil Direct |
| | | | | | LabViro123 | Submission |
| AL157109 | 11.7 | 2015 | Zikavirus | BR | BRlUFBN | Zika Vims Outbreak; Bahia; Brazil Direct |
| | | | | | LabViro/17 | Submission |
| AL157108 | 11.7 | 2015 | Zikavirus | BR | BRlUFBN | Zika Vims Outbreak; Bahia; Brazil Direct |
| | | | | | LabViro/18 | Submission |
| AL157107 | 11.7 | 2015 | Zikavirus | unknown | BRlUFBN | Zika Vims Outbreak; Bahia; Brazil Direct |
| | | | | | LabVirolEx 1 | Submission |
| AKN44264 | 13.6 | 2015 | Zikavirus | unknown | 15098 | First report of autochthonous transmission of |
| | | | | | | Zika vims in Brazil Direct Submission |
| AKN44263 | 13.6 | 2015 | Zikavirus | unknown | 15095 | First report of autochthonous transmission of |
| | | | | | | Zika vims in Brazil Direct Submission |

A large increase in Replikin concentration was identified between 2014 and 2015. Additionally, the highest Replikin count of a single isolate in 2014 as compared to 2015 increased. Further, a remarkable increase was observed from 2014 to 2015 in the percentage of isolates having a Replikin count of greater than 4.0.

In 2014, 66 accession numbers were identified. Five isolates had a Replikin concentration of greater than 4.0 resulting in 7.6% of isolates having a concentration greater than 4.0. The highest Replikin concentration identified in 2014 was 6.9 Replikin sequences per 100 amino acid residues.

In 2015, twelve accession numbers were identified. Of those twelve sequences, nine had a Replikin concentration of greater than 4.0 resulting in 75% of isolates having a concentration of greater than 4.0. The highest Replikin concentration identified in 2015 was 13.6.

These marked changes correlate with the outbreak of ZIKV in 2015 and 2016.

FIG. 1 similarly illustrates that a rise in Replikin concentration occurs before the ZIKV outbreak and increase in virulence and morbidity (as demonstrated by a spike in cases of microcephaly) in the virus population.

It is noteworthy that isolates from the Easter Islands around 2012 and 2013 reflected a Replikin concentration of only about 4 and much lower virulence and morbidity. In contrast, the 2015 outbreak in Brazil follows an increase in Replikin concentration to around 8 in May and as much as 13.6 in October of 2015. Following this increase, certain reported cases of microcephaly rose from about 150 per year to likely more than 4000.

The data in FIG. 1 reflects a rather small number of samples (n=179) from 1947 to 2015 and yet it directly illustrates the correlation between rise in Replikin concentration and outbreak. This speaks to a need for constant surveillance of Replikin concentration in isolates of virus.

### Example 2

### Conservation of KHWLVHKEWFH (SEQ ID NO: 1) in ZIKV

The peptide KHWLVHKEWFH (SEQ ID NO: 1) was identified as conserved in reported isolates of ZIKV from the following years in the listed accession numbers. All occurrences identified by applicants in a given year at NCBI PubMed database website are listed.

| | |
|---|---|
| 1947 | AEN75263 position 499 |
| 1963 | AHL43457 position 73 |
| 1966 | AEN75264 position 499 |
| 1968 | AEN75265 position 493 |
| 1969 | AHL43453 position 79 |
| 1976 | AHF49783 position 499 |
| 1979 | AHL43456 position 79, AHL43455 position 79, AHL43454 |
| | position 79 |
| 1980 | AHL43442 position 79 |
| 1981 | AHL43452 position 79 |
| 1984 | AEN75266 position 499 |
| 1990 | AHL43467 position 79, AHL43466 position 79, AHL43465 |
| | position 79, AHL43463 position 79 |
| 1991 | AHL43461 position 79 |
| 1996 | AHL43459 position 79 |
| 1997 | AHL43449 position 79, AHL43448 position 79, AHL43447 |
| | position 79, AHL43446 position 79, AHL43445 position 79, |
| | AHL43444 position 79 |
| 1998 | AHL43443 position 79, AHL43441 position 79 |
| 1999 | AHL43468 position 79, AHL43460 position 79, AHL43458 |
| | position 79 |
| 2000 | AHL43440 position 79 |
| 2001 | AHL43439 position 79, AHL43437 position 79, AAK91609 |
| | position 85 |
| 2002 | AHL43451 position 79, AHL43450 position 79 |
| 2005 | AAV34151 position 495 |
| 2007 | AHL16749 position 90, ACD75819 position 499 |
| 2009 | YP 002790881 position 495, ABI54475 position 493. |
| 2010 | AFD30972 position 499 |
| 2013 | AHL37808 position 462, AHZ13508 position 499 |
| 2014 | AHF49785 position 499, AHF49784 position 493, BAP47441 |
| | position 499, AIC06934 position 209, AHL43505 position 499, |
| | AHL43504 position 499, AHL43503 position 499, AHL43501 |
| | position 499, AHL43500 position 499 |
| 2015 | ALU33341 position 499 |

SEQ ID NO: 1 is a Replikin peptide sequence identified as conserved in ZIKV across many years. Replikin peptide sequences are associated with rapid replication and virulence in ZIKV (see FIG. 1) and are understood to be immunogenic because of the positioning of the lysine residues in the sequence. The histidine in the Replikin peptide sequence likewise is associated with rapid replication and virulence. As a result, SEQ ID NO: 1 and homologues of SEQ ID NO: 1 are useful in a vaccine or in blocking composition for preventing and/or treating ZIKV infection. SEQ ID NO: 1 may be used alone or in combination with other Replikin sequences or homologues of Replikin sequences. SEQ ID NO: 1 may be covalently linked to other Replikin sequences, such as anyone or more of SEQ ID NO(s): 2-7. Peptidomimetic versions of SEQ ID NO: 1 or homologues thereof may likewise be used for immunogenic or blocking compositions. Poly-N-substituted glycines, D-peptides, or betapeptides may be constructed to function as SEQ ID NO: 1.

### Example 3

### Conservation of KHWLVHK (SEQ ID NO: 2) in ZIKV

The peptide KHWLVHK (SEQ ID NO: 2) was identified as conserved in reported isolates of ZIKV from the following years in the listed accession numbers. All occurrences identified by applicants in a given year at NCBI PubMed database website are listed.

| | |
|---|---|
| 1947 | AEN75263 position 499 |
| 1963 | AHL43457 position 73 |
| 1966 | AEN75264 position 499 |
| 1968 | AEN75265 position 493 |
| 1969 | AHL43453 position 79 |
| 1976 | AHF49783 position 499 |
| 1979 | AHL43456 position 79, AHL43455 position 79, AHL43454 |
| | position 79 |
| 1980 | AHL43442 position 79 |
| 1981 | AHL43452 position 79 |
| 1984 | AEN75266 position 499 |
| 1990 | AHL43467 position 79, AHL43466 position 79, AHL43465 |
| | position 79, AHL43463 position 79 |
| 1991 | AHL43461 position 79 |
| 1996 | AHL43459 position 79 |
| 1997 | AHL43449 position 79, AHL43448 position 79, AHL43447 |
| | position 79, AHL43446 position 79, AHL43445 position 79, |
| | AHL43444 position 79 |
| 1998 | AHL43443 position 79, AHL43441 position 79 |
| 1999 | AHL43468 position 79, AHL43460 position 79, AHL43458 |
| | position 79 |
| 2000 | AHL43440 position 79 |
| 2001 | AHL43439 position 79, AHL43437 position 79, AAK91609 |
| | position 85 |
| 2002 | AHL43451 position 79, AHL43450 position 79 |
| 2005 | AAV34151 position 495 |
| 2007 | AHL16749 position 90, ACD75819 position 499 |
| 2009 | YP 002790881 position 495, ABI54475 position 493. |
| 2010 | AFD30972 position 499 |
| 2013 | AHL37808 position 462, AHZ13508 position 499 |
| 2014 | AHF49785 position 499, AHF49784 position 493, BAP47441 |
| | position 499, AIC06934 position 209, AHL43505 position 499, |
| | AHL43504 position 499, AHL43503 position 499, AHL43501 |
| | position 499, AHL43500 position 499 |
| 2015 | ALU33341 position 499 |

SEQ ID NO: 2 is a Replikin peptide sequence identified as conserved in ZIKV across many years. Replikin peptide sequences are associated with rapid replication and virulence in ZIKV (see FIG. 1) and are understood to be immunogenic because of the positioning of the lysine residues in the sequence. The histidine in the Replikin peptide sequence likewise is associated with rapid replication and virulence. As a result, SEQ ID NO: 2 and homologues of SEQ ID NO: 2 are useful in a vaccine or in blocking composition for preventing and/or treating ZIKV infection. SEQ ID NO: 2 may be used alone or in combination with other Replikin sequences or homologues of Replikin sequences. SEQ ID NO: 2 may be covalently linked to other Replikin sequences, such as anyone or more of SEQ ID NO(s): 1 and 3-7. Peptidomimetic versions of SEQ ID NO: 2 or homologues thereof may likewise be used for immunogenic or blocking compositions. Poly-N-substituted glycines, D-peptides, or betapeptides may be constructed to function as SEQ ID NO: 2.

### Example 4

### Conservation of KEALVEFKDAH (SEQ ID NO: 3) in ZIKV

The peptide KHWLVHK (SEQ ID NO: 3) was identified as conserved in reported isolates of ZIKV from the following years in the listed accession numbers. All occurrences identified by applicants in a given year at NCBI PubMed database website are listed.

| | |
|---|---|
| 1947 | AEN75263 position 529 |
| 1963 | AHL43457 position 103 |
| 1966 | AEN75264 position 529 |
| 1968 | AEN75265 position 523 |
| 1976 | AHF49783 position 529 |
| 1979 | AHL43456 position 109, AHL43455 position 109, AHL43454 |
| | position 109 |
| 1980 | AHL43442 position 109 |
| 1981 | AHL43452 position 109 |
| 1984 | AEN75266 position 529 |
| 1990 | AHL43467 position 109, AHL43466 position 109, AHL43465 |
| | position 109, AHL43464 position 109, AHL43463 position 109, |
| | AHL43462 position 109 |
| 1991 | AHL43461 position 109 |
| 1996 | AHL43459 position 109 |
| 1997 | AHL43449 position 109, AHL43448 position 109, AHL43447 |
| | position 109, AHL43446 position 109, AHL43445 position 109, |
| | AHL43444 position 109 |
| 1998 | AHL43443 position 109, AHL43441 position 109 |
| 1999 | AHL43468 position 109, AHL43460 position 109, AHL43458 |
| | position 109 |
| 2000 | AHL43440 position 109 |
| 2001 | AHL43439 position 109, AHL43438 position 109, AHL43437 |
| | position 109, AAK91609 position 115 |
| 2002 | AHL43451 position 109, AHL43450 position 109 |
| 2005 | AAV34151 position 525 |
| 2007 | AHL16749 position 120, ACD75819 position 529 |
| 2009 | yP 002790881 position 525, ABI54475 position 523 |
| 2010 | AFD30972 position 529 |
| 2013 | AHL37808 position 492, AHZ13508 position 529 |
| 2014 | AHF49785 position 529, AHF49784 position 523, BAP47441 |
| | position 529, AIC06934 position 239, AHL43505 position 529, |
| | AHL43504 position 529, AHL43503 position 529, AHL43502 |
| | position 529, AHL43501 position 529, AHL43500 position 529 |
| 2015 | ALU33341 position 529, AKN44264 position 9, AKN44263 |
| | position 9 |

SEQ ID NO: 3 is a Replikin peptide sequence identified as conserved in ZIKV across many years. Replikin peptide sequences are associated with rapid replication and virulence in ZIKV (see FIG. 1) and are understood to be immunogenic because of the positioning of the lysine residues in the sequence. The histidine in the Replikin peptide sequence likewise is associated with rapid replication and virulence. As a result, SEQ ID NO: 3 and homologues of SEQ ID NO: 3 are useful in a vaccine or in blocking composition for preventing and/or treating ZIKV infection. SEQ ID NO: 3 may be used alone or in combination with other Replikin sequences or homologues of Replikin sequences. SEQ ID NO: 3 may be covalently linked to other Replikin sequences, such as anyone or more of SEQ ID NO(s): 1,2, and 4-7. Peptidomimetic versions of SEQ ID NO: 3 or homologues thereof may likewise be used for immunogenic or blocking compositions. Poly-N-substituted glycines, D-peptides, or beta-peptides may be constructed to function as SEQ ID NO: 3.

### Example 5

### Conservation of KGRLSSGHLK (SEQ ID NO: 4) in ZIKV

The peptide KGRLSSGHLK (SEQ ID NO: 4) was identified as conserved in reported isolates of ZIKV from the following years in the listed accession numbers. All occurrences identified by applicants in a given year at NCBI PubMed database website are listed.

| | |
|---|---|
| 1963 | AHL43457 position 145 |
| 1966 | AEN75264 position 571 |
| 1968 | AEN75265 position 565 |
| 2001 | AAK91609 position 157 |
| 2007 | ACD75819 position 571 |
| 2010 | AFD30972 position 571 |
| 2013 | AHL37808 position 534, AHZ13508 position 571 |
| 2014 | AIC06934 position 281 |
| 2015 | ALU33341 position 571, ALI57109 position 35, ALI57108 |
| | position 35, ALI57107 position 35, ALI57106 position 35, |
| | AKN44264 position 51, AKN44263 position 51 |

SEQ ID NO: 4 is a Replikin peptide sequence identified as conserved in ZIKV across many years. Replikin peptide sequences are associated with rapid replication and virulence in ZIKV (see FIG. 1) and are understood to be immunogenic because of the positioning of the lysine residues in the sequence. The histidine in the Replikin peptide sequence likewise is associated with rapid replication and virulence. As a result, SEQ ID NO: 4 and homologues of SEQ ID NO: 4 are useful in a vaccine or in blocking composition for preventing and/or treating ZIKV infection. SEQ ID NO: 4 may be used alone or in combination with other

Replikin sequences or homologues of Replikin sequences. SEQ ID NO: 4 may be covalently linked to other Replikin sequences, such as anyone or more of SEQ ID NO(s): 1,2,3, and 5-7. Peptidomimetic versions of SEQ ID NO: 4 or homologues thereof may likewise be used for immunogenic or blocking compositions. Poly-N-substituted glycines, D-peptides, or beta-peptides may be constructed to function as SEQ ID NO: 4.

### Example 6

### Conservation of HLKCRLKMDK (SEQ ID NO: 5) in ZIKV

The peptide HLKCRLKMDK (SEQ ID NO: 5) was identified as conserved in reported isolates of ZIKV from the following years in the listed accession numbers. All occurrences identified by applicants in a given year at NCBI PubMed database website are listed.

| | |
|---|---|
| 1947 | AEN75263 position 578 |
| 1963 | AHL43457 position 152 |
| 1966 | AEN75264 position 578 |
| 1968 | AEN75265 position 572 |
| 1969 | AHL43453 position 158 |
| 1976 | AHF49783 position 578 |
| 1979 | AHL43456 position 158, AHL43455 position 158, AHL43454 |
| | position 158 |
| 1981 | AHL43425 position 158 |
| 1984 | AEN75266 position 578 |
| 1990 | AHL43467 position 158, AHL43466 position 158, AHL43465 |
| | position 158, AHL43464 position 158, AHL43463 position 158, |
| | AHL43462 position 158 |
| 1991 | AHL43461 position 158 |
| 1996 | AHL43459 position 158 |
| 1997 | AHL43449 position 158, AHL43448 position 158, AHL43447 |
| | position 158, AHL43446 position 158, AHL43445 position 158, |
| | AHL43444 position 158 |
| 1999 | AHL43468 position 158, AHL43460 position 158, AHL43458 |
| | position 158 |
| 2001 | AAK91609 position 164 |
| 2002 | AHL43451 position 158, AHL43450 position 158 |
| 2005 | AAV34156 position 570, AAV34151 position 574 |
| 2007 | AHL16749 position 169, ACD75819 position 578 |
| 2009 | YP_002790882 position 570, YP_002790881 position 574, |
| | ABI54475 position 572 |
| 2010 | AFD30972 position 578 |
| 2013 | AHL37808 position 541, AHZ13508 position 578 |
| 2014 | AHF49785 position 578, AHF49784 position 572, AJD81420 |
| | position 282, BAP47441 position 578, AIC06934 position 288, |
| | AHL43505 position 578, AHL43504 position 578, AHL43503 |
| | position 578, AHL43502 position 578, AHL43501 position 578, |
| | AHL43500 position 578 |
| 2015 | ALU33341 position 578, ALI57109 position 42, ALI57108 |
| | position 42, ALI57107 position 42, ALI57106 position 42, |
| | AKN44264 position 58, |

SEQ ID NO: 5 is a Replikin peptide sequence identified as conserved in ZIKV across many years. Replikin peptide sequences are associated with rapid replication and virulence in ZIKV (see FIG. 1) and are understood to be immunogenic because of the positioning of the lysine residues in the sequence. The histidine in the Replikin peptide sequence likewise is associated with rapid replication and virulence. As a result, SEQ ID NO: 5 and homologues of SEQ ID NO: 5 are useful in a vaccine or in blocking composition for preventing and/or treating ZIKV infection. SEQ ID NO: 5 may be used alone or in combination with other Replikin sequences or homologues of Replikin sequences. SEQ ID NO:5 maybe covalently linked to other Replikin sequences, such as anyone or more of SEQ ID NO(s): 1-4, 6, and 7. Peptidomimetic versions of SEQ ID NO: 5 or homologues thereof may likewise be used for immunogenic or blocking compositions. Poly-N-substituted glycines, D-peptides, or betapeptides may be constructed to function as SEQ ID NO: 5.

### Example 7

### Conservation of HWNNKEALVEFK (SEQ ID NO: 6) in ZIKV

The peptide HWNNKEALVEFK (SEQ ID NO: 6) was identified as conserved in reported isolates of ZIKV from the following years in the listed accession numbers. All occurrences identified by applicants in a given year at NCBI PubMed database website are listed.

| | |
|---|---|
| 1947 | AEN75263 position 525 |
| 1963 | AHL43457 position 99 |
| 1966 | AEN75264 position 525 |
| 1968 | AEN75265 position 519 |
| 1976 | AHF49783 position 525 |
| 1979 | AHL43456 position 105, AHL43455 position 105, AHL43454 |
| | position 105 |
| 1980 | AHL43442 position 105 |
| 1981 | AHL43452 position 105 |
| 1984 | AEN75266 position 525 |
| 1990 | AHL43467 position 105, AHL43466 position 105, AHL43465 |
| | position 105, AHL43464 position 105, AHL43463 position 105, |
| | AHL43462 position 105 |
| 1991 | AHL43461 position 105 |
| 1996 | AHL43459 position 105 |
| 1997 | AHL43449 position 105, AHL43448 position 105, AHL43447 |
| | position 105, AHL43446 position 105, AHL43445 position 105, |
| | AHL43444 position 105 |
| 1998 | AHL43443 position 105, AHL43441 position 105 |
| 1999 | AHL43468 position 105, AHL43460 position 105, AHL43458 |
| | position 105 |
| 2000 | AHL43440 position 105 |
| 2001 | AHL43439 position 105, AHL43438 position 105, AHL43437 |
| | position 105, AAK91609 position III |
| 2002 | AHL43451 position 105, AHL43450 position 105 |
| 2005 | AAV34151 position 521 |
| 2007 | AHL16749 position 116, ACD75819 position 525 |
| 2009 | yP_002790881 position 521, ABI54475 position 519 |
| 2010 | AFD30972 position 525 |
| 2013 | AHL37808 position 488, AHZ13508 position 525 |
| 2014 | AHF49785 position 525, AHF49784 position 519, BAP47441 |
| | position 525, AIC06934 position 235, AHL43505 position 525, |
| | AHL43504 position 525, AHL43503 position 525, AHL43502 |
| | position 525, AHL43501 position 525, AHL43500 position 525 |
| 2015 | ALU33341 position 525, AKN44264 position 5, AKN44263 |
| | position 5 |

SEQ ID NO: 6 is a Replikin peptide sequence identified as conserved in ZIKV across many years. Replikin peptide sequences are associated with rapid replication and virulence in ZIKV (see FIG. 1) and are understood to be immunogenic because of the positioning of the lysine residues in the sequence. The histidine in the Replikin peptide sequence likewise is associated with rapid replication and virulence. As a result, SEQ ID NO: 6 and homologues of SEQ ID NO: 6 are useful in a vaccine or in blocking composition for preventing and/or treating ZIKV infection. SEQ ID NO: 6 may be used alone or in combination with other Replikin sequences or homologues of Replikin sequences. SEQ ID NO: 6 may be covalently linked to other Replikin sequences, such as anyone or more of SEQ ID NO(s): 1-5 and 7. Peptidomimetic versions of SEQ ID NO: 6 or homologues thereof may likewise be used for immunogenic or blocking compositions. Poly-N-substituted glycines, D-peptides, or beta-peptides may be constructed to function as SEQ ID NO: 6.

### Example 8

### Conservation of HRTLALAVIKYTYQNK (SEQ ID NO: 7) in ZIKV

The peptide HRTLALAVIKYTYQNK (SEQ ID NO: 7) was identified as conserved in reported isolates of ZIKV from the following years in the listed accession numbers. All occurrences identified by applicants in a given year at NCBI PubMed database website are listed.

| | |
|---|---|
| 1947 | AEN75263 position 3082 |
| 1996 | AHL43490 position 80 |
| 1997 | AAC58803 position 101 |
| 1999 | AHL43489 position 80, AHL43488 position 80, AHL43471 |
| | position 80. |
| 2005 | AAV34151 position 3078 |
| 2009 | YP_002790881 position 3078, ABI54475 position 3076 |
| 2014 | BAP47441 position 3082, AHL43505 position 3082, AHL43504 |
| | position 3082, AHL43503 position 3082 |

SEQ ID NO: 7 is a Replikin peptide sequence identified as conserved in ZIKV across many years. Replikin peptide sequences are associated with rapid replication and virulence in ZIKV (see FIG. 1) and are understood to be immunogenic because of the positioning of the lysine residues in the sequence. The histidine in the Replikin peptide sequence likewise is associated with rapid replication and virulence. As a result, SEQ ID NO: 6 and homologues of SEQ ID NO: 7 are useful in a vaccine or in blocking composition for preventing and/or treating ZIKV infection. SEQ ID NO: 7 may be used alone or in combination with other Replikin sequences or homologues of Replikin sequences. SEQ ID NO: 7 may be covalently linked to other Replikin sequences, such as anyone or more of SEQ ID NO(s): 1-6. Peptidomimetic versions of SEQ ID NO: 7 or homologues thereof may likewise be used for immunogenic or blocking compositions. Poly-N-substituted glycines, D-peptides, or betapeptides may be constructed to function as SEQ ID NO: 7.

### Example 9

### Synthetic Replikin Vaccine Against ZKV Using Selected Conserved Peptides

A synthetic Replikin vaccine containing approximately equal-parts-by-weight of the following seven peptides was designed and prepared for use against ZIKV:

(SEQ ID NO: 1)
KHWLVHKEWFH
(SEQ ID NO: 2)
KHWLVHK
(SEQ ID NO: 3)
KHWLVHK
(SEQ ID NO: 4)
KGRLSSGHLK
(SEQ ID NO: 5)
HLKCRLKMDK
(SEQ ID NO: 6)
HWNNKEALVEFK
(SEQ ID NO: 7)
HRTLALAVIKYTYQNK

The peptides are synthesized by solid-state synthesis and lyophilized. The lyophilized peptides are mixed together. A sufficient amount of mixture is dissolved in deionized water to prepare a solution having a concentration of 4 g/L.

A single rabbit is prepared for testing of the vaccine. The rabbit is bled and tested for antibodies to the mixture of peptides at various stages before, during, and after administration of the vaccine. Antibody response is determined via ELISA. The ELISA plate is prepared by adherence of the mixture of the peptides to the surface of the wells of the plate. Concentration of binding antibodies on the plate is determined via optical density.

The single rabbit is tested for antibody response prior to administration of the vaccine (Pre-bleed) on day 0.

A 25 μL aliquot of 4 g/L aqueous peptide mixture is administered to the rabbit (a 100 μg dose) intranasally on day 1. A 25 μL aliquot of 4 g/L aqueous peptide mixture is administered to the rabbit (a 100 μg dose) intranasally on day 15.

A 25 μL aliquot of 4 g/L aqueous peptide mixture is administered to the rabbit (a 100 μg dose) intranasally on day 22 along with a 50 μL aliquot of 4 g/L aqueous peptide mixture (a 200 μg dose) given via intramuscular administration. Blood is drawn from the rabbit on day 22 and ELISA tested for antibody response against the mixture of peptides (1^{st} Bleed).

Blood is drawn from the rabbit on day 36 and ELISA tested for an antibody response against the mixture of peptides (2^{nd} Bleed).

A 25 μL aliquot of 4 g/L aqueous peptide mixture is administered to the rabbit (a 100 μg dose) intranasally on day 50 along with a 50 μL aliquot of 4 g/L aqueous peptide mixture (a 200 μg dose) given via intramuscular administration.

Blood is drawn from the rabbit on day 57 and ELISA tested for antibody response against the mixture of peptides (3^{rd} Bleed).

A 25 μL aliquot of 4 g/L aqueous peptide mixture is administered to the rabbit (a 100 μg dose) intranasally on day 80 along with a 50 μL aliquot of 4 g/L aqueous peptide mixture (a 200 μg dose) intramuscular administration.

Blood is drawn from the rabbit on day 87 and ELISA tested for antibody response against the mixture of peptides (4^{th} Bleed).

The peptides of the vaccine are conserved Replikin peptides of ZIKY. FIG. 1 demonstrates that Replikin sequences of ZIKV are structurally and functionally related to rapid replication and virulence in ZIKV and are conserved across strains of ZIKV, including most virulent strains. Because Replikin sequences are structurally and functionally related to rapid replication and virulence, targeting Replikin structures provides a mechanism for reducing rapid replication and virulence. The vaccine is therefore useful for preventing and treating ZIKV infection by targeting these replication mechanisms.

The vaccine of the example (as well as individual peptides of the vaccine or various mixtures of the individual peptides of the vaccine) is likewise useful as an immunogenic composition for stimulating the immune system of a subject against ZIKV, against an infection of an ZIKV, or to prevent a ZIKV infection. Antibodies produced from the immune response are likewise useful for diagnosing ZIKV contamination and infection.

The vaccine of the example (as well as individual peptides of the vaccine or various mixtures of the individual peptides of the vaccine) is useful as a blocking composition to block replication of virus within infected cells.

### Example 10

### Computer Methods for Determining Virulence of ZIKV

A prediction of expansion or retraction of virulence of ZIKV population may be performed by a processor. A prediction may be output to a user or display. Likewise, a particular Replikin peptide or Replikin Peak Gene within an isolate or population of isolates of KIKV may be predicted to be expanding or retracting in replication or virulence and this prediction may be output to a user or display. A machine-readable storage medium may contain executable instructions that, when executed by a processor, cause the processor to provide sufficient data to a user, a printout, or a display such that the user or a user of the printout or display may predict expansion or retraction of population of ZIKY. A process for prediction may comprise: comparing a Replikin concentration of at least one first isolate of ZIKV with a Replikin concentration of at least one second isolate of ZIKV; and predicting the population of the first isolate to be expanding if the Replikin concentration of the first isolate is greater than the Replikin concentration of the second isolate. In another embodiment, predicting the population of the first isolate to be expanding if the Replikin concentration of the first isolate is greater than eight or greater than ten Replikin sequences per 100 amino acid residues.

A computer system may include a processor coupled to a network, and a memory coupled to a processor, wherein the memory contains a plurality of instruction to perform the methods of prediction discussed herein.

A user of outputted data from a processor, storage medium, machine-readable medium, or computer system may include any person or any machine that records or analyzes the outputted data. A display or printout may include any mechanism by which data is outputted so that any person or any machine may record or analyze the outputted data, including a printed document, a visual impulse, an aural impulse, or any other perceivable impulse, a computer monitor, a set of numbers, or any other display or printout of data including a digital recording medium. A computer program product may have computer program logic arranged to put into effect a method of predicting an outbreak of ZIKY. The program may be contained in a signal or non-transitory signal.

### Example 11

### Trivalent Vaccine Based on 100% Identical Replikin Sequence Identified in ZIKV, DEN, and JEV

On Jan. 26, 2016, Replikins, Ltd. reported that Replikins sequences in the Zika gene have been found to be at the highest concentrations observed since 1947 and that retrospective genomic analysis of the Zika gene shows marked changes in the Zika genome over the past year. Previous outbreaks of Zika, in Africa and the Pacific, recorded on PubMed since 1947, never had Replikin counts greater than 9.6. Zika counts in Brazil this year have reached 13.6. Furthermore, 75% of the current Zika gene counts are greater than 4.0, the count in the recent Easter Island outbreak in 2014. Replikins has completed discovery on a synthetic genomic-based peptide vaccine-blocker for Zika virus based on the conserved Replikin sequences in the Zika genome.

Based on the sharing of sequences among ZIKV, DEN, and JEV, a trivalent synthetic Replikins vaccine and blocker was designed and manufactured by Replikins, Ltd. Testing is now underway in animals.

The new trivalent vaccine and blocker was designed based on the finding of the inventors following examination of all sequences on PubMed as of February 2016, that: 1) identical and homologous gene Replikins sequences occur in, that is, are shared by, different Flaviviruses, 2) that some of these gene Replikins are conserved back to 1947, and 3) evidence has been found of sharing, for example, due to independent appearance or possible sequential transfer between 2010 and 2014 of an identical Replikin structure between the Japanese Encephalitis virus gene and the Dengue virus gene, then first to appear in the Zika gene in 2013, and to peak in the Zika gene in 2014, giving one year early warning before the outbreak in 2015.

The appearance in time of this particular Replikin sequence in the Japanese Encephalitis virus and Dengue virus, then in the Zika virus is of interest with regard to the increase 20-fold in fetal microcephaly, increase in Guillain Barre Syndrome, and other nervous system disorders reported to be associated with Zika virus infection.

### Example 12

### SEQ ID NO: 5 Shared Among JEV, DEN, and ZIKV

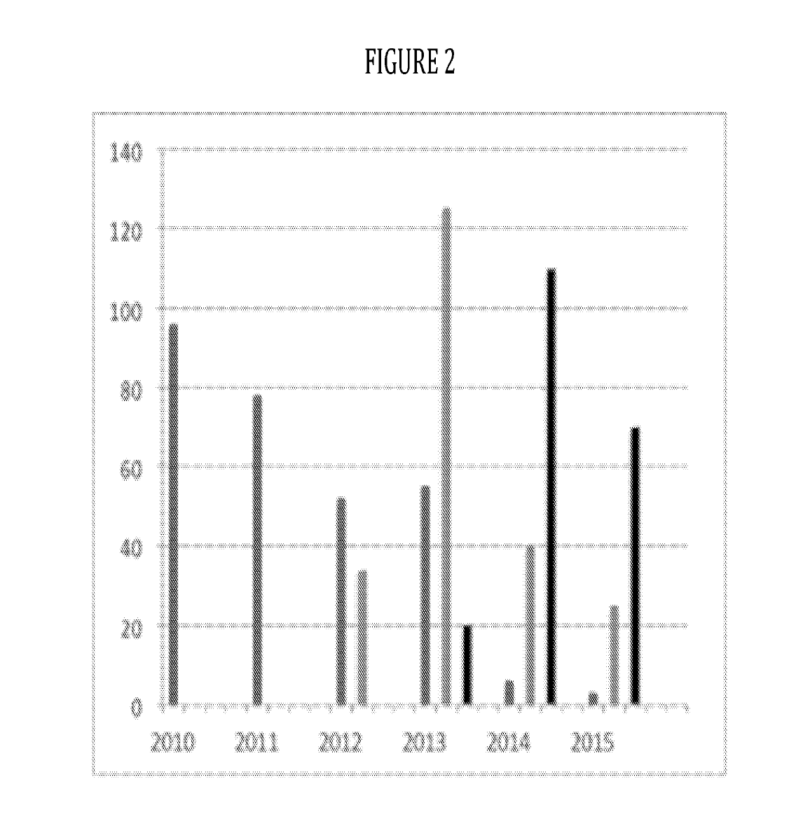

Table 2 below provides the number of isolates in which SEQ ID NO: 5 appeared in a given year in three Flavivirus genes (JEV, DEN, and ZIKV) in specimens reported at the NCBI PubMed database. The data are illustrated in FIG. 2. The number of isolates of ZIKV identified with SEQ ID NO: 5 is multiplied by 10 in FIG. 2 so that the ZIKV data appears in the same visual range as the JEV and DEN data. As is understood by the artisan, the number of ZIKV isolates reported to the NCBI PubMed database has been relatively small compared to the number of JEV and DEN isolates.

TABLE 2

| Year | JEV | DEN | ZIKV |
|---|---|---|---|
| 2010 | 96 | 144 | |
| 2011 | 78 | 46 | |
| 2012 | 52 | 34 | |
| 2013 | 55 | 125 | 2 |
| 2014 | 6 | 40 | 11 |
| 2015 | 3 | 25 | 7 |

1. A composition comprising at least one protein, protein fragment, polypeptide, peptide, or peptides corresponding to the full sequence or at least one functional fragment of thereof an analog, or homologue of a Replikin sequence selected from of any one of SEQ ID NO(s): 1-7.

2. The composition of claim 1 comprising a mixture of two or more peptides individually or bonded by a covalent link.

3. The composition of claim 1 where composition comprises units of a Poly-N-substituted glycine, D-peptide, or beta-peptide.

4. The composition of claim 2 where the covalent link is a peptide bond, peptoid bond, an Ahx spacer, or PEGylation.

5. The composition of claim 4 where the units are linked by PEGylation, peptoid bonds, or Ahx spacers.

6. An isolated, chemically-synthesized, or recombinantly-generated binding molecule that specifically binds to at least one sequence of SEQ ID NO(s): 1-7.

7. The binding molecule of claim 6 comprising a Replikin-specific antibody where the antibody is fc-optimized, selected using phage display, or produced from a microorganism.

8. The binding molecule of claim 6 comprising a nucleic acid sequence that is antisense to a nucleic acid that encodes for any Replikin sequence in or identified in an isolate of a Flavivirus.

9. A method of providing passive immunity in a patient suffering from an Flavivirus infection comprising administering to the patient at least one isolated, chemically-synthesized, or recombinantly-generated binding molecule of claim 6.

10. A vaccine against a Flavivirus comprising the composition of claim 1.

11. The vaccine of claim 10 where the vaccine is multi-valent.

12. The vaccine of claim 10 against Japanese encephalitis virus (JEV), dengue virus, and Zika virus.

13. The vaccine of claim 10 where the peptide has the same, analogous, or homologous amino acid sequence as at least one protein, protein fragment, polypeptide or peptide identified in a relatively virulent strain of a Flavivirus up to seven days, one month, six months, one year, two years, or three years prior to making said vaccine.

14. The vaccine of claim 10 contains one or more Replikin sequence or homologue thereof, selected from SEQ ID NO(s): 1-7, where the Replikin sequence or homologue thereof, shared between two or more Flaviviruses is used as a specific target, without the need for differential diagnosis between individual Flaviviruses.

15. Claim 14 where the two or more Flaviviruses are Japanese Encephalitis virus, Dengue virus, and Zika virus.

16. A method of prediction comprising:
a. obtaining a plurality of isolates of a Flavivirus wherein at least one of said isolates is isolated about six months to about 3 years later than at least one other of said isolates;
b. analyzing the amino acid sequence of at least one protein or protein fragment in each isolate of the plurality of isolates for the presence and concentration of Replikin sequences;
c. comparing the concentrations of Replikin sequences in the at least one protein or protein fragment in each isolate of the plurality of isolates one to another;
d. identifying a change in the concentration of Replikin sequences in said plurality of isolates over at least one time period of about six months or greater;
e. predicting the virulence of an Flavivirus based on regression analysis of the change in the concentration of Replikin sequences;
f. determine and report a portion of a pathogen predicted to be an expanding Flavivirus or predicted to increase in virulence or morbidity wherein said portion or its antimere, not limited to antibody, may be employed as a therapeutic or diagnostic compound.
g. determining if a here the portion of a pathogen is a Replikin peptide or plurality of Replikin peptides or any other structure or portion of said genome of said pathogen including a Replikin Peak Gene.
thereby predicting the progression or waning of outbreaks of a Flavivirus, and determining effective therapeutic agents, within about one month to about three years following said identified increase in the concentration of Replikin sequences.

17. Claim 16 where the Replikin concentration is a mean Replikin concentration of a plurality of isolates with standard deviation from the mean and the standard deviation from the mean is greater than the standard deviation from the mean Replikin concentration of a plurality of other isolates.

18. The method of claim 16 where the population of an isolate when compared to a previous population is predicted to be expanding if the Replikin concentration of the isolate is greater than three Replikin sequences per 100 amino acid residues.

19. A vaccine of a portion of a pathogen or its antimere, not limited to an antibody determined to predict the progression or waning of outbreaks of a Flavivirus, and determining effective therapeutic agents, within about one month to about three years following said identified increase in the concentration of Replikin sequences by
a. obtaining a plurality of isolates of a Flavivirus wherein at least one of said isolates is isolated about six months to about 3 years later than at least one other of said isolates;
b. analyzing the amino acid sequence of at least one protein or protein fragment in each isolate of the plurality of isolates for the presence and concentration of Replikin sequences;
c. comparing the concentrations of Replikin sequences in the at least one protein or protein fragment in each isolate of the plurality of isolates one to another;
d. identifying a change in the concentration of Replikin sequences in said plurality of isolates over at least one time period of about six months or greater;
e. predicting the virulence of an Flavivirus based on the change in the concentration of Replikin sequences;
f. determine and report a portion of a pathogen predicted to be an expanding Flavivirus or predicted to increase in virulence or morbidity wherein said portion or its antimere, not limited to antibody, may be employed as a therapeutic or diagnostic compound.
g. determining if a here the portion of a pathogen is a Replikin peptide or plurality of Replikin peptides or any other structure or portion of said genome of said pathogen including a Replikin Peak Gene.
manufactured following the differentiation between relatively more virulent and relatively less virulent forms of Flavivirus, where the vaccine is manufactured following prediction of an outbreak of Flavivirus following identification of a more virulent form of a Flavivirus.


SEQUENCE LISTING (9 sequences)

SEQ ID NO: 1 - protein, 11 aa, ZIKA
KHWLVHKEWFH

SEQ ID NO: 2 - protein, 7 aa, ZIKA
KHWLVHK

SEQ ID NO: 3 - protein, 11 aa, ZIKA
KEALVEFKDAH

SEQ ID NO: 4 - protein, 10 aa, ZIKA
KGRLSSGHLK

SEQ ID NO: 5 - protein, 10 aa, ZIKA
HLKCRLKMDK

SEQ ID NO: 6 - protein, 12 aa, ZIKA
HWNNKEALVEFK

SEQ ID NO: 7 - protein, 16 aa, ZIKA
HRTLALAVIKYTYQNK

SEQ ID NO: 8 - protein, 7 aa, ZIKA
KHATVLK

SEQ ID NO: 9 - protein, 10 aa, ZIKA
KVKKHATVLK